SEQUENCE LISTING (18 sequences)

SEQ ID NO: 1 - DNA, 3739 bp, synthetic construct
  misc_feature 1..3739: note = Nucleotide sequence of the transforming DNA from pPB0014
  source 1..3739: mol_type = other DNA
aagcttcaagtgcgtgcgttacagtgttaccaacaacagtctaacctacccctttcggtcattctgccctttggcaagagttcagaatgaagtgtgcttgcacatcgagctagtgctgtgagcgaagacaaggaagtccccactcacccacgtggccagattctatcttttttcagattgcaagggccacgcccagcgaaccccgcgatggggccgagccatgcccgacatctcgacatcttcatatgataaggcgcttcaaagtgcaatttttgtgcatggcatcaattaggagagtgcttgaacaccagcccatcttccaccggggaaggaccgtcgaaatgcctctgcagacggccaccgtctgatcgctgcctgtcccgaggtgacggcgatgtcgtccttatcccaaacaatcgttcgaagacctttcttttgttcgctcaacccaccgaggagaccgtctggattccatgctgctgtgacgcctagccccctgagaccctccaagtgggcggtcccctccctagcccccagcctctctgacgtggcagatgcctccgcggaagcaaatcaggatcgcagggagggctcctacgagcagcccctggtccaacgccaggtgcctagggggaaaggagggcagaggggcttgaggcgagcctggcccaggcagggcttccatggtcagtcgtggcagtgccatgacagccgaagcccaacgcgacaccgtgggtgcagcatgcgtggacggaaacattggcaatgccttgccccattggccccccaggcccggaaacgggacgatcagcaggaccccttgtccagcctcctccccacggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgattgattggaactcacaaagcggcccacggcttcgaacgtcccgtgtcaattgcgcggggtgtgccagagtttctgcgccaccgatgctcaccctagggggggatgccctttgacattcatgtgtgcctgcatgcacgtttgtatcagtctcaccacaccttgaagatttttgggagggggggggaagtcggaatggaaacgagctccgcgattgtcagatggtggagtgggtggatggccctgctccgaggagctttctagggcgcgaacttggcccttctcccctctgatgcagtgtggggggacgcggtgtgctatttctccgagggccgcccaactagggtggggcgggcatacgcccgcgtcgacaggtgggtgtggcctcgaggtgttgagaggagtgttatgtcgacagccaaagtggagactgattgaaccctactccaggtgctatcttgggagcacactgcgcccaccgtggctggactgcccgaattccaaccttggtgcccagaaacagggcaaagccggtcatcagtgcagcatgagactcaagctccctagctcatgaccgttggcataggcagaagctgcggcagcacctggtggaggcctgccaggcaaacggtgtcaccttccagccgggggaggtagtggatgtgggcgtgaagaacggcacagcctcggtcacctgccaagatggctccgtcctgactgcgaggtgggcgctgtgcatggcatttgttggcacgagtctgcatctctgaagctgctgggtagcgtcagagcagtggagtcaacagcacacagctctggcggtgctcagggaacatacatcgcactgtttcctggagttgctggccctctgtggggcaaccaggaccccccgacgcatgcatgccccctcgcacatcccgcacaggctggtgaccctggcctccggcgcggcggcggggcgcttcctcaagtacgagagaagctt

SEQ ID NO: 2 - DNA, 4611 bp, synthetic construct
  misc_feature 1..4611: note = Nucleotide sequence of the transforming DNA from pPB0038
  source 1..4611: mol_type = other DNA
aagcttcaagtgcgtgcgttacagtgttaccaacaacagtccaacctaaccctttcggtcattctgtcctttggcaagggctcagaatgaagtgtgcctgcacatcgagctagtgctgtgagcgaagacaaggaagtccccactcacccacgtggccagattttatcttttttcagattgcaagggccacgcccagcgaaccccgcgatggggccgagccatgcccgacatctcgacatcttcatatgacaaggcgcttcaaagtgcaatttatgtgcatggcatcgattaggagagtggttgaacaccagcccatcttccaccggggaaggaccgtcgaaatgcctctgcagacggccaccgtctgatcgctgcctgtcccgaggtgacggcgatgtcgtccttatcccaaacaatcgttcgaagacctttcttttgttcgctcaacccaccgaggagaccgtctggattccatgccgctgtgacgcctagccccctgagaccctccaagtgggcggtcccctccctagcccccagcctctctgacgtggcagatgcctccgcggaagcaaatcaggatcgcagggagggctcctacgagcagcccctggtccaacgccaggtgcctagggggaaaggagggcagggggccttgaggcgagcctggcccaggcagggcttccatggtcagtcgtggcagtgccatgacagccgaagcccaccgcgacaccgtgggtgcagcatgcgtggacggaaacattggcaatgccttgccccattggacccccaggcccggaaacgggacgatcagcaggaccccctgtccagcctcctccccacggtacctcccgctttttaattgagcccctttcgtcgctgaatcagcgaaagcaccgcgaaacaatgcctgtcccgtccatgcatctcaacagcctcatgcaaggtttgcacaagcaagaccattctgatctgggaacttgtaggtgttgtatgggggaggttgtgctcttgaatcaagtggtatcacgtttccggaacaccccgaaacgtgcatgggcttattgcgatgagagcatttcccaccgcgattgtctcacgcgcatttcggagaaggtttgcagaacactccaggacatgaaatgccttgtcacgtatgaaccatctcccacggccttgaaaagatcgctcgacttccattctagatggtgcaaaaccctacgactcaagaaggtgccaccgactcaggcattgggcacggcgggcagggagaagagaggagttgatcaaaactgctcgatcacgttcccccatggcgatccgagcagcacatgatgcatcgaggtggcgccgttgcaaaggagttgcgcatgggtcgaagcagggagaaggaaacggcgaggcgtgccgcgggggtgaattcagagtcaaatctgcgcctgccccggcgctcctgacggggattaacccccacgactgtatccatcgacactcgtctcgggggaataaaagcggcgacccagctccagaggcgcaatccttctcacaatctgtttaactttcaacaaagtataagtcaattcaacttgacacaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgagtcctggcgaccctgctcccctgacccctgttcccctgcgctgcttctccccggtgacatccgacctgctgcaaaattcccgttcctgcacaacacttgcctgaccgagggtcgggtcgcgaagtaaaagccacaatcaacaccccaggcacattaagagtgcacagcatgacgcagcatagggtttgtgtcggaggaagggggtcgagtcgcgttggcgagggggtggtcacgatgaccacatctgcgggataattgaatcctcaggggaaaataccagtctctgcttccaggtgctccggagctccgcgattgtcagatggtggagtgggtggatggccctgctccagaggagcgttctagggcgcaaacttggcactttcccccctctgatgcagtgtggggggacgcggtgtgctatttctccgagggccgcccaaccagggtggggcgggcatatgcccgcgtcgacaggtgggtgtggcctcgaggtgttgagaggagggttatgtcgacagccaaagtggagactgagtgaaccctactccaggtgctgtcgtgggagcgcactgcgcccaccgtggctggactgctcgtattccaaccttggtgcccagaaacagggcaaagccggtcatcagtgcagcatgagactcaagctccctaactcatgaccgttggcataggcagaagctgcggcagcacctggtggaggcctgccaggcaaacggtgtcaccttccagccgggggaggtagtggatgtgggcgtgaagaacggcacagcctcggtcacctgccgagacggctccgtcctgactgcgaggtgggcgctgtgcatggcacttgttggcacgagtctgcatccctgaagctgctgggtagcgtcagagcagtggcgtcaacagcacacagctctggcggtgcccagggaacatacatcgctctgtttcctggagttgcgggccctctgtgtggcagccagggccccccgacgcatgcatgcctcctcgcacatcccgcacaggctggtgaccctggcctccggcgcggcggcggggcgcttcctcaagtacgagagaagctt

SEQ ID NO: 3 - DNA, 2081 bp, synthetic construct
  misc_feature 1..2081: note = Nucleotide sequence of the CrBKT1 expression module from pPB0120
  source 1..2081: mol_type = other DNA
ggtacccttgcagtgccccaaaaactggctaccacctaacaattctcacgcagttttatcctctgcactttgatgtcagctttttgattcgtctgcgtacattacagcgttgagtggccagcaggaaggagaccgcggtccgagacgagtctgagggcgcgctctcgcaacttggattccggatttcttaccctgcatcgacctcggcctggagtcgatcagaaattgtcattgccagattgcctggcgaggacgggtgatatactcaaggcgttgcatcgcccacaaaacacacacttatctgcaagggagttactgcatcaggctctgctcaacagctcgtgacatcgatcgttcagctccccagcaggtgcgtgtccgcatggagcacccctcccgagacacctgcgttgggtgtcggaggagctcacatgccagggaggtgcccacattgcaccacgcgaccgcgaaataggcagacttcgggcatcctgtcatcgcatgtccgctggccgggaatcatggcctccccaccaggcgtcacgcgctgcccacctccctccccttgctgcgcagggcaccgcgttcctgtggagagccgaccacatgggccccggcatccagcccacctccgcccgcccctgctcccgcaccaagcactcccgcttcgccctgctggccgccgccctgaccgcccgccgcgtgaagcagttcaccaagcagttccgctcccgccgcatggccgaggacatcctgaagctgtggcagcgccagtaccacctgccccgcgaggactccgacaagcgcaccctgcgcgagcgcgtgcacctgtaccgccccccccgctccgacctgggcggcatcgccgtggccgtgaccgtgatcgccctgtgggccaccctgttcgtgtacggcctgtggttcgtgaagctgccctgggccctgaaggtgggcgagaccgccacctcctgggccaccatcgccgccgtgttcttctccctggagttcctgtacaccggcctgttcatcaccacccacgacgccatgcacggcaccatcgccctgcgcaaccgccgcctgaacgacttcctgggccagctcgcgatctccctgtacgcctggttcgactactccgtgctgcaccgcaagcactgggagcaccacaaccacaccggcgagccccgcgtggaccccgacttccaccgcggcaaccccaacctggccgtgtggttcgcccagttcatggtgtcctacatgaccctgtcccagttcctgaagatcgccgtgtggtccaacctgctgctgctggccggcgcccccctggccaaccagctgctgttcatgaccgccgcccccatcctgtccgccttccgcctgttctactacggcacctacgtgccccaccaccccgagaagggccacaccggcgccatgccctggcaggtgtcccgcacctcctccgcctcccgcctgcagtccttcctgacctgctaccacttcgacctccactgggagcaccaccgctggccctacgccccctggtgggagctgcccaagtgccgccagatcgcccgcggcgccgccctggcctgagcggaggccttggaaatattcgcgtcacgcgaggagtaggctctgctggtcggccctggatacgctgactcttcaagcagtggggcaccacacccaccttttgccaagggcaaggagtcggaagggggcggggctgccatgcacccctgacgggcatggccgttccgcgagggcgccaactgcggcggcctgcccgctggctcgtgcccccctacccccaccattgcctggagcgtttccatccccaaatcacattccatccaagttgtatcactatgcccctttggctctatacactcacggcctgaggtcccttctcggccgtggcggcacacgcccaaccccccaccatactctttccatacactgcaatgcttcgagcctgcctgccacctgctctgcttgtctcccctcccttcccttgaggttttccaatgcagtaagagaagtcgacgtgcatggacagatgattgagagatgagactagt

SEQ ID NO: 4 - DNA, 981 bp, synthetic construct
  misc_feature 1..981: note = Nucleotide sequence of the ApSAD2tp_CrBKT1 expression module from pPB0123
  source 1..981: mol_type = other DNA
atggccaccgcatccactttctcggcgttcaatgcccgctgcggcgacctgcgtcgctcggcgggctccgggccccggcgcccagcgaggcccctccccgtgcgcgctgccatccgctcccgccgcatggccgaggacatcctgaagctgtggcagcgccagtaccacctgccccgcgaggactccgacaagcgcaccctgcgcgagcgcgtgcacctgtaccgccccccccgctccgacctgggcggcatcgccgtggccgtgaccgtgatcgccctgtgggccaccctgttcgtgtacggcctgtggttcgtgaagctgccctgggccctgaaggtgggcgagaccgccacctcctgggccaccatcgccgccgtgttcttctccctggagttcctgtacaccggcctgttcatcaccacccacgacgccatgcacggcaccatcgccctgcgcaaccgccgcctgaacgacttcctgggccagctcgcgatctccctgtacgcctggttcgactactccgtgctgcaccgcaagcactgggagcaccacaaccacaccggcgagccccgcgtggaccccgacttccaccgcggcaaccccaacctggccgtgtggttcgcccagttcatggtgtcctacatgaccctgtcccagttcctgaagatcgccgtgtggtccaacctgctgctgctggccggcgcccccctggccaaccagctgctgttcatgaccgccgcccccatcctgtccgccttccgcctgttctactacggcacctacgtgccccaccaccccgagaagggccacaccggcgccatgccctggcaggtgtcccgcacctcctccgcctcccgcctgcagtccttcctgacctgctaccacttcgacctccactgggagcaccaccgctggccctacgccccctggtgggagctgcccaagtgccgccagatcgcccgcggcgccgccctggcctga

SEQ ID NO: 5 - protein, 328 aa, synthetic construct
  REGION 1..328: note = Amino acid sequence of native CrBKT1
MGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALA

SEQ ID NO: 6 - protein, 326 aa, synthetic construct
  REGION 1..326: note = Amino acid sequence of chimeric ApSAD2tp_CrBKT1
MATASTFSAFNARCGDLRRSAGSGPRRPARPLPVRAAIRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALA

SEQ ID NO: 7 - DNA, 4637 bp, synthetic construct
  misc_feature 1..4637: note = Nucleotide sequence of the transforming DNA from pPB0065
  source 1..4637: mol_type = other DNA
aagctttcactgctccattcacacccaatttcccaccgccgcacccgctcccaggtgggaggatgggagggaacccagctgggagccggaggagcatgtgaccccggacctgatcgagctatatgccgccagggacgctcgagcacgggatgtcaaggccgcggctgacaaggctggcaagggtaggacacctgcgttgaggcaaacgctggagacggccggtctcagcatgtgatcctgtacttgctgtgacaaggtggacaatgcagtacgaggtgtaccaggctaagtattcctagtataccggcacgccagatcatctgtacaggagtctgtgcaggagtgaaaaggtaagagccagagccatgaccggcgaccatccatgtcccgtcactcggatgcactggctgacatcggcggcaggtcatccaacggtcctgaatgatcgagaggaagctgcccgatttcaaaacgcccccccacgtcgccctccatggccgcacagcatgctcagcacaggttgctgcgtgtcctcacacaaactgctcctttaaagcgatcaactttccagggcatggggcactcgtactgacaatcacccacattcgtatacctttgacgtcattattttttcgccccaacgcggttgccatcccgagttgtacctccgcggctaccatacccctgtcttctggccctcaccgtcgctcgcaggcgggatccagcgcagccagtctgaatacttttacacaacatagtacgtaacgcgcattaggcccccaataccagcagctggctccagcatgggcaagggtgcgcggcaggaggaattctcccgctttttaattgagcccctttcgtcgctgaatcagcgaaagcaccgcgaaacaatgcctgtcccgtccatgcatctcaacagcctcatgcaaggtttgcacaagcaagaccattctgatctgggaacttgtaggtgttgtatgggggaggttgtgctcttgaatcaagtggtatcacgtttccggaacaccccgaaacgtgcatgggcttattgcgatgagagcatttcccaccgcgattgtctcacgcgcatttcggagaaggtttgcagaacactccaggacatgaaatgccttgtcacgtatgaaccatctcccacggccttgaaaagatcgctcgacttccattctagatggtgcaaaaccctacgactcaagaaggtgccaccgactcaggcattgggcacggcgggcagggagaagagaggagttgatcaaaactgctcgatcacgttcccccatggcgatccgagcagcacatgatgcatcgaggtggcgccgttgcaaaggagttgcgcatgggtcgaagcagggagaaggaaacggcgaggcgtgccgcgggggtgaattcagagtcaaatctgcgcctgccccggcgctcctgacggggattaacccccacgactgtatccatcgacactcgtctcgggggaataaaagcggcgacccagctccagaggcgcaatccttctcacaatctgtttaactttcaacaaagtataagtcaattcaacttgacacaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgagtcctggcgaccctgctcccctgacccctgttcccctgcgctgcttctccccggtgacatccgacctgctgcaaaattcccgttcctgcacaacacttgcctgaccgagggtcgggtcgcgaagtaaaagccacaatcaacaccccaggcacattaagagtgcacagcatgacgcagcatagggtttgtgtcggaggaagggggtcgagtcgcgttggcgagggggtggtcacgatgaccacatctgcgggataattgaatcctcaggggaaaataccagtctctgcttccaggtgctccggagctccccaggcgagtcaatcagttgtgtcatgagattgatctgcctgttgcagatcccccgacccgctgccggcccctctgccgtgcgacaccccttgccctggggtgtgcctcttgtcctgcatcgcacacctcctccgccggaccttcaccccctcccacctcgacacaagcaggtgtgggacgtgatagtggtgggcgcgggcgtggccggcgcggcgctggcgcatcagcagggcttggacggccgacgcgtgctgctcctcgagcgggatctggcccagcccgaccgcatcgtgggcgagctgctgcagcctggcggcgtgctggccctggagcgcctgggcctgggcggcgccgtggacggcatcgacgcgcagcccgtggtcgggtactgcatgttcaagggcgggcgcgaggcgtgcatcgcctaccccacccccgccgagctggggggtccagcggctgcggctgcggcatgcaggggccccactggaagcgccagcgccgcgcccgccggcgacgcccccgtcacgggcttctccttccacaacgggcgattcgtgcagcggctgcgcgccgcggcggcggctgcgcccggggtcacgctgcgtcgcggcacggtgcgcgcgctggtggatgacgccggcgcggactgggaggaggggcgcgtggtgacgggcgtgcggtaccgcgcgggcgacggcggcgagcgcgtggcactgggccacctcaccgtggtctgcgacggcatgtactcggccctgcggtccaagctggcggtgcccgacctgcgcacgccctcccacttcatcaagctt

SEQ ID NO: 8 - DNA, 3767 bp, synthetic construct
  misc_feature 1..3767: note = Nucleotide sequence of the transforming DNA from pPB0077
  source 1..3767: mol_type = other DNA
aagctttcactgctccattcacacccaatttcccaccgccgcacccgctcccaggtgggaggatgggagggaacccagctgggagccagaggagcatgtgaccccggacctgatcaagctatatgccgccagggacgctcgagcacgggatgtcaaggccgcggctgacaaggctggcaagggcaggacacctgcgccgaggcaaacgctggagacggccggtctcagcatgtgatcctgtacttgctgtgacaaggtggacaatgcagtacgagttgtaccaggctaagtatccctagtataccggcacgccagatcatctgtacaggagtctgtgcaggagtaaaaaggcaacagccagagccatgaccggcgactatccacatcccgtcactccgatgcactggctgacatcggcggcaggtcgtccaacggtcctgaatgatcgagaggaagctgcccgatttcaccccccccccccacgtcgccctccatggccgcacagcatgctcagcacaggttgctgcgtgtcctcacacaaactgctcctttaaagcgatcaactttccagggcatggggcactcgtactgacaatcacccacattcgtatacctttgacgtcatttttttttcgccccaacgcggttgccatcccgagttgtacctccgcggctaccatacccctgtcttctggccctcaccgtcgctcgcaggcgggatccagcgcagccagtctgaatacttttacacaacatagtacgtaacgcgcattaggcccccaataccagcagctggctccagcatgggcaagggtgcgcggcaggaggaattcctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgattgattggaactcacaaagcggcccacggcttcgaacgtcccgtgtcaattgcgcggggtgtgccagagtttctgcgccaccgatgctcaccctagggggggatgccctttgacattcatgtgtgcctgcatgcacgtttgtatcagtctcaccacaccttgaagatttttgggagggggggggaagtcggaatggaaacgagctccccaggcgagtcaatcagttgtgtcatgagattgatctgcctgttgcagatcccccgacccgctgccggcccctctgccgtgcgacaccccttgccctggggtgtgcctcttgtcctgcatcgcacacctcctccgccggaccttcaccccctcccacctcgacacaagcaggtgtgggacgtgatagtggtgggcgcgggcgtggccggcgcggcgctggcgcatcagcagggcttggacggccgacgcgtgctgctcctcgagcgggatctggcccagcccgaccgcatcgtgggcgagctgctgcagcctggcggcgtgctggccctggagcgcctgggcctgggcggcgccgtggacggcatcgacgcgcagcccgtggtcgggtactgcatgttcaagggcgggcgcgaggcgtgcatcgcctaccccacccccgccgagctggggggtccagcggctgcggctgcggcatgcaggggccccactggaagcgccagcgccgcgcccgccggcgacgcccccgtcacgggcttctccttccacaacgggcgattcgtgcagcggctgcgcgccgcggcggcggctgcgcccggggtcacgctgcgtcgcggcacggtgcgcgcgctggtggatgacgccggcgcggactgggaggaggggcgcgtggtgacgggcgtgcggtaccgcgcgggcgacggcggcgagcgcgtggcactgggccacctcaccgtggtctgcgacggcatgtactcggccctgcggtccaagctggcggtgcccgacctgcgcacgccctcccacttcatcaagctt

SEQ ID NO: 9 - protein, 730 aa, synthetic construct
  REGION 1..730: note = Amino acid sequence of A. protothecoides 1-deoxy-D-xylulose 5-phosphate synthase (DXS)
MQFSLAGMNTRALQTGARPSLPAARPSRRVRPARRSAPCPVARTMGGGEEQPSSAEGVAWDKISTDELADWAGAGPPTPLLDTVAFPVHIKNFNSRQLQQLCKELRADLIHTVAKTGGHLGSSLGVVELTVALHHVFNTPEDRIVWDVGHQAYIHKMLTGRRARMHTIRQQGGLSGFTRRAESVYDPFGAGHSSTSVSAALGMAVGRDRKGRANNCIAVIGDGAITGGMAYEAMNHAGFLDTNMIVILNDNQQVSLPTQYNGKNQEPVGALSSALARLQANRQLRELREIAKGVTKQLPDVIQNATAKIDEYARGMISGTGSTLFEELGFYYIGPVDGHNMQDLVDVLSEIKATETVGPVLLHVVTQKGRGYTPAETASDRMHGVVQYDTLTGKQKKGSGGPQSYTNYFADALVAEAKRDARVLGIHAAMGGGTGMNRFEAAFPDRVFDTGIAEQHAVTFAAGLATEGLVPFVAIYSTFLQRGYDQIVHDVSLQSLPVRFALDRAGNVGADGATHAGAFDVTYLACLPNMVVMAPSNEAELVHAVATAAAIDDRPSAFRFPRGNGLGVDLAAAGVTDDLKGQPMEVGRGVVRRGGADVALLGYGTCVNACLAAADLLAAQGVSATVVDARFCKPLDTALVRRMAAEHPVMITVEEGSIGGFAAHVMQFLALEGLLDGKLKFRPMTLPDRYIEHGTQAEQMAEAGLTASHIAGTALSVMGVKRDAPSIFST

SEQ ID NO: 10 - protein, 453 aa, synthetic construct
  REGION 1..453: note = Amino acid sequence of A. protothecoides 1-deoxy-D-xylulose 5-phosphate reductoisomerase (DXR)
MRCSAQLNTRGPTLPNSARPRTCRVVSASAAPVPSAWPGRVVLPEKSASRTGPKKFSLLGSTGSIGTQTLDIVAEHPDRFQVVSLAAGGNVALLAEQIARFSPSLVSVRDSGGARALEAALDAAGVDRRPEIQIGAAGIDAVAAHPEADACVTGIVGCAGLRPTMAAIEAGKDICLANKETLIAGGPTVLPAAAKHGVSILPADSEHSAIFQCLQGLPEGGLRRIILTASGGAFRDLPVSELPKVTVADALKHPNWAMGKKITIDSATLMNKGLEVIEAHYLFGASYDNIDIVIHPQSIVHSMIETQDSSVLAQLGWPDMRLPILYTMSWPERVPCSEVTWPRLDFVKAGNLTFRQPDHAKYPAMELAYSAGRAGGTMTGVMSAANEAAVELFLEEAIGYLDIVPVVEAACEAHRVELVERPSLEEIVHYDQWARRHVRESVAKRAPAAVPAL

SEQ ID NO: 11 - protein, 359 aa, synthetic construct
  REGION 1..359: note = Amino acid sequence of A. protothecoides farnesyl diphosphate synthase (FDPS)
MAAVVEAGHAASKQKTEAHQTKQEFLAVFEKLRDELLEDSILAGQPESSKDWLRTMLDYNVPHGKLNRGMAVLDVLLAARGGDVTEKEREAANVLGWCIELLQAYFLVADDIMDSSLTRRGQPCWYRQPHVGMVAINDGIILESCIYRLLKLHFRAHPAYVHLLELFHDTTHRTAHGQLLDTTTAPPGGVDLTRYTEGTYLRIVTYKTAFYTIYLPVACGLALAGVTDEASLALAEDLSVRMGRYFQIQDDVLDAFGEPEVIGKVGTDIQDSKCSWLVVRALAVASAEQREAIKANYGRDDAEAVEAVKAVYRELDLPAAFAAYEQESYDGLVQAIEGQDKFPPAVFMGILAKIYKRTK

SEQ ID NO: 12 - protein, 458 aa, synthetic construct
  REGION 1..458: note = Amino acid sequence of A. protothecoides squalene synthase (SQS)
MGKLGELLSHPDEIIPMAAMYLAARRAAVLPHDPDLAFCYSMLNKVSRSFAIVIQQLPEQLRDAVCVFYLVLRALDTVEDDMAIDQAEKVPILLSFHEKTYEKDWSMKCGHGHYVELMEQYPVVCAAFQGLEPQYQEVITDICRRMGAGMAEFIVKEVETVKDYDLYCHYVAGLVGVGLSNLFAGSGLESEDFASLHELSNGMGLFLQKTNIIRDYLEDIMEEPAPRMFWPKEIWGKHGDSLEDFKDPENAEAAVACLNDMIADALRHVDASLDYMQRLRNRPIFRFCAVPQIMAIGTLAACFDNPSVFTGVVKMRRGQTAKIMHDVEDYADLLAYFRAFGQALAAKARAARGKGAESVGRAAERVVAGCSAALADLSRAENARMAAAARRPLSLPARALLLVAALLYLFLAWRAEGVRRWLGVDSPPAAHKLDYYNQIVASMFLGYSLFAVGTGRRP

SEQ ID NO: 13 - DNA, 5161 bp, synthetic construct
  misc_feature 1..5161: note = Nucleotide sequence of the transforming DNA from pPB0039
  source 1..5161: mol_type = other DNA
aagcttgcacagtcagtcgtcatccacgaagtcgcgcccgtctgtccaccggggtctcctgaacgcagcaatctcctcctgagtgtatgagcccgtggccgggagtttgtatgcagggcgaggcaaggacgaccatgccgggagaaacccaaggtgacgaagtgacattgtgctcgatcactccatgcactgcctcactcgcccatgtaccttggtcatgtactccccagtctgcatcttggtgttcctgttcagctcgcggagctcctccaggcgctgctcgtcgcggcggtcccgcgagatgtaaaaggcagggacacccacccccagggccactcccagtagagccccccctacagcaaccagggtgtccggatcgctaaagtcgatgttgatcgcgcggcacggtggcattatcgggcgtggatggccccatgatcggtgcaacgaaggcgccctcatggcaggtccgcatggtcgtccattgcaggggatacccgctcgcactttcgttgacaataacatcctcgtatagttggagaaaggatttgtgatctgtctctggaggcccttaaagtcctgcccctcctctgctggaacctgacctctcatgcccctgcgccacgcccccggatctgatatggctctgatatgggtggctcgtacctcttggctaggcgacccccctaagcacgcgtgcgggccagggcacaacattatattttgccctctccttcgtcaacgctcatttttttggaatactaacgtttaaaagctctcgggtacctcccgctttttaattgagcccctttcgtcgctgaatcagcgaaagcaccgcgaaacaatgcctgtcccgtccatgcatctcaacagcctcatgcaaggtttgcacaagcaagaccattctgatctgggaacttgtaggtgttgtatgggggaggttgtgctcttgaatcaagtggtatcacgtttccggaacaccccgaaacgtgcatgggcttattgcgatgagagcatttcccaccgcgattgtctcacgcgcatttcggagaaggtttgcagaacactccaggacatgaaatgccttgtcacgtatgaaccatctcccacggccttgaaaagatcgctcgacttccattctagatggtgcaaaaccctacgactcaagaaggtgccaccgactcaggcattgggcacggcgggcagggagaagagaggagttgatcaaaactgctcgatcacgttcccccatggcgatccgagcagcacatgatgcatcgaggtggcgccgttgcaaaggagttgcgcatgggtcgaagcagggagaaggaaacggcgaggcgtgccgcgggggtgaattcagagtcaaatctgcgcctgccccggcgctcctgacggggattaacccccacgactgtatccatcgacactcgtctcgggggaataaaagcggcgacccagctccagaggcgcaatccttctcacaatctgtttaactttcaacaaagtataagtcaattcaacttgacacaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgagtcctggcgaccctgctcccctgacccctgttcccctgcgctgcttctccccggtgacatccgacctgctgcaaaattcccgttcctgcacaacacttgcctgaccgagggtcgggtcgcgaagtaaaagccacaatcaacaccccaggcacattaagagtgcacagcatgacgcagcatagggtttgtgtcggaggaagggggtcgagtcgcgttggcgagggggtggtcacgatgaccacatctgcgggataattgaatcctcaggggaaaataccagtctctgcttccaggtgctccgactagtcttgcagtgccccaaaaactggctaccacctaacaattctcacgcagttttatcctctgcactttgatgtcagctttttgattcgtctgcgtacattacagcgttgagtggccagcaggaaggagaccgcggtccgagacgagtctgagggcgcgctctcgcaacttggattccggatttcttaccctgcatcgacctcggcctggagtcgatcagaaattgtcattgccagattgcctggcgaggacgggtgatatactcaaggcgttgcatcgcccacaaaacacacacttatctgcaagggagttactgcatcaggctctgctcaacagctcgtgacatcgatcgttcagctccccagcaggtgcgtgtccgcatggagcacccctcccgagacacctgcgttgggtgtcggaggagctcacatgccagggaggtgcccacattgcaccacgcgaccgcgaaataggcagacttcgggcatcctgtcatcgcatgtccgctggccgggaatcatggcctccccaccaggcgtcacgcgctgcccacctccctccccttgctgcgcagggcaccgcgttcctgtggagagccgaccacatggtcctgtgcgctggtagtttgagctgcagggcgccgccttcaggcggtgctaggttggagcgggggtcccccttcgtgcgcctgggccaccatgcccgcccacaagctcgcagacggatgtcagacctcgtaataaggtccatcgcagcccctgctccgcccccgctgcgacctgcatctcagcctcaagccccacctgcatcagagaccttcacggtgagtcatgatcgaggtcggcccctgcagcgctcgtgctccggaaacgacccgctactcaatccctgaaccatgaatacttcaggggggccgcgaactggccaaccgccctcccttctccctccaagacatccgcaacgccatcccggcggagtgctggaagaaggacaccttcaagtccttcgcctacctggcgctagatgtaggaatcgttgctgccctggccgtgacagcacatgccgtcaactcgccatggctctggcccttctactggctggcgcaggggaccatgttctgggccctgtttgtcgtcggccacgactggtgcggttggagggggtaatctggcgaccctgcagggcatgcagtggggacaagagcatcgccaggctccgccttgcctgctgatcccagcccgacttggctggacaatagatgctgtcggggacatgccgcagtgcatgccacaatgggccccttcaacaactgaccaccactatgacccatattccctgcagcggccaccagagcttcagcacaaacaagcagctgaatgacgtggaagctt

SEQ ID NO: 14 - DNA, 774 bp, synthetic construct
  misc_feature 1..774: note = Nucleotide sequence of the ApFATA promoter in pPB0041
  source 1..774: mol_type = other DNA
ggaatcccgcctccgagatgaagccgtggttggcacggaggaggccgctgcgggccagagtgttcttctgctgcacgtcctccggctttggtggctcgctgggcttgggtgcggccatgagctgcagtgcaagtgtacatataggtcaatcttatgacccggcactaccaatgatgatcaacaccgagcggccctctgtgttgtgcttgcctctttaccttcactgcgtactgctgcaggagcttcatgaggatcacactgacggtcagggggatcagcacccagtcccggacatcccgatccagtacgaggtcctggctgaccatgatggtaggtgaagttgggccctgggaggagcgctagaggagcctcggggcaaagatcaccctactctgacgtggctggctcaatcacccatccctcccctttgaagtcggctctcagtttgcgttgtttcgaaatcgagccacaatcgaatatacactacctaaaggctctcaccacctggcgtacctcggaatgcccatcagcccaaacacatgagaaaaggcgcgcgcggttcgaccccagtccgtcgattgacgcagtggggagctccattctgtcagctcttgggtggccaggtcgctgacagattggcacatacaggaccctgccgacccgttcctccagcactttgtgaatttaagcagcgcattagatcgtcgatggcttagagaaccccgcgcctgctcccccatctccctttcacacgtttgaacacccggaccggcc

SEQ ID NO: 15 - DNA, 5651 bp, synthetic construct
  misc_feature 1..5651: note = Nucleotide sequence of the transforming DNA from pPB0118
  source 1..5651: mol_type = other DNA
aagcttagcatactcctattctgacaatgtcacagtcggtctgccaggcgatagtggctttgctgtcagactcggccccggactctcccctgaactgcgacgccgggaatctgttgagaggaggcgatctgcgagggttcgcctccatggcccgcatgtacaccatcgagtatgccatgaagcgatgatgtctgtgaaaatgatgttcagaattcattatatactcatgtttttgtgtaaatgctgtgtcgacttaagttaccgagctggctgacagagacaatcttcaggtcaaatgttggcaccaatgatcgcgacgatcgttcaggggttatcaagtcagatctgaacgaaaaccagaaatcaaatttgccaaagcgcatgtttgtatgtcgagaattatcatgcgggtgactggctcgctaattctggcatggaaggatgccacatcgaattgatccggggagactaacacttgtcagaattgcaatgtgccatattccagatatcccagccggcccttctataaaccacctgcgggctcagatacctacgaagaggctcagataactcaaggacgtgcattcgaattatccctgccgcgcggaaacatcagaccaggtgcggatgctgagcgtcgagttgggtgcttgatagaccttcaccttgatctgaggttcccgtccccagagcactcgaatctccggcatcttacaggcaaaccgcaaacagtaaataatggcgagcaccatcaccatgggtacccttgcagtgccccaaaaactggctaccacctaacaattctcacgcagttttatcctctgcactttgatgtcagctttttgattcgtctgcgtacattacagcgttgagtggccagcaggaaggagaccgcggtccgagacgagtctgagggcgcgctctcgcaacttggattccggatttcttaccctgcatcgacctcggcctggagtcgatcagaaattgtcattgccagattgcctggcgaggacgggtgatatactcaaggcgttgcatcgcccacaaaacacacacttatctgcaagggagttactgcatcaggctctgctcaacagctcgtgacatcgatcgttcagctccccagcaggtgcgtgtccgcatggagcacccctcccgagacacctgcgttgggtgtcggaggagctcacatgccagggaggtgcccacattgcaccacgcgaccgcgaaataggcagacttcgggcatcctgtcatcgcatgtccgctggccgggaatcatggcctccccaccaggcgtcacgcgctgcccacctccctccccttgctgcgcagggcaccgcgttcctgtggagagccgaccacatgtccgccgccgccgccgagaccgacgtgtccctgcgccgccgctccaactccctgaacggcaaccacaccaacggcgtggccatcgacggcaccctggacaacaacaaccgccgcgtgggcgacaccaacacccacatggacatctccgccaagaagaccgacaacggctacgccaacggcgtgggcggcggcggctggcgctccaaggcctccttcaccacctggaccgcccgcgacatcgtgtacgtggtgcgctaccactggatcccctgcatgttcgccgccggcctgctgttcttcatgggcgtggagtacaccctgcagatgatccccgcccgctccgagcccttcgacctgggcttcgtggtgacccgctccctgaaccgcgtgctggcctcctcccccgacctgaacaccgtgctggccgccctgaacaccgtgttcgtgggcatgcagaccacctacatcgtgtggacctggctggtggagggccgcgcccgcgccaccatcgccgccctgttcatgttcacctgccgcggcatcctgggctactccacccagctgcccctgccccaggacttcctgggctccggcgtggacttccccgtgggcaacgtgtccttcttcctgttcttctccggccacgtggccggctccatgatcgcctccctggacatgcgccgcatgcagcgcctgcgcctggccatggtgttcgacatcctgaacgtgctgcagtccatccgcctgctgggcacccgcggccactacaccatcgacctggccgtgggcgtgggcgccggcatcctgttcgactccctggccggcaagtacgaggagatgatgtccaagcgccacctgggcaccggcttctccctgatctccaaggactccctggtgaactgagcggaggccttggaaatattcgcgtcacgcgaggagtaggctctgctggtcggccctggatacgctgactcttcaagcagtggggcaccacacccaccttttgccaagggcaaggagtcggaagggggcggggctgccatgcacccctgacgggcatggccgttccgcgagggcgccaactgcggcggcctgcccgctggctcgtgcccccctacccccaccattgcctggagcgtttccatccccaaatcacattccatccaagttgtatcactatgcccctttggctctatacactcacggcctgaggtcccttctcggccgtggcggcacacgcccaaccccccaccatactctttccatacactgcaatgcttcgagcctgcctgccacctgctctgcttgtctcccctcccttcccttgaggttttccaatgcagtaagagaagtcgacgtgcatggacagatgattgagagatgagactagtctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgattgattggaactcacaaagcggcccacggcttcgaacgtcccgtgtcaattgcgcggggtgtgccagagtttctgcgccaccgatgctcaccctagggggggatgccctttgacattcatgtgtgcctgcatgcacgtttgtatcagtctcaccacaccttgaagatttttgggagggggggggaagtcggaatggaaacctcgagcaacgctacgcaactcccttcgatggcttcaagtacggagatgtgggcatccaggattcgcatgtgctgcttcagccctcctcatgccactagcactcatttttcgactcccggattgccaggttcaagggcatcaaggagtcggagatcagccgcgccatgacctcccgctacttcgaggacctaaacgtcaatgccgaggtgcttttgcatatatttacagctaattatgatgggtgtggtgcgcgatatgcttgcaaggtctccggtgagctaatgatcggcacatcccttccgcgcatccgcaggtcgatgtcgtcattgttggggctgggtctgcgggcctctcgtgcgcctatgagctgagcaagcacccggatgtcaaggtatgggctgagcagggcacatcctcagatgatgttgctgtaattgcaattgaaacttgcggttgttcccagcacagcctcaatcaatcatgtgtgctgcgttggaaacgctatgataccccagccttcaacatggggcagggatatcgtttacacctgcttgaaccccccgcaacaggtggccatcatcgagcagggcgtcgcccctgggggtggagcgtggctggggggtcagctcttctcggctatgtgtgtgagtctaggcacggggacgggtggactgaagcaagggttgggcgcagggtgttgatatccatgtgttggacattctcgttgggaaaacaagatgtgtgtatttagtgctatctcggtggctgcattccaagctt

SEQ ID NO: 16 - DNA, 5526 bp, synthetic construct
  misc_feature 1..5526: note = Nucleotide sequence of the transforming DNA from pPB0142
  source 1..5526: mol_type = other DNA
aagcttagcatactcctattctgacaatgtcacagtcggtctgccaggcgatagtggctttgctgtcagactcggccccggactctcccctgaactgcgacgccgggaatctgttgagaggaggcgatctgcgagggttcgcctccatggcccgcatgtacaccatcgagtatgccatgaagcgatgatgtctgtgaaaatgatgttcagaattcattatatactcatgtttttgtgtaaatgctgtgtcgacttaagttaccgagctggctgacagagacaatcttcaggtcaaatgttggcaccaatgatcgcgacgatcgttcaggggttatcaagtcagatctgaacgaaaaccagaaatcaaatttgccaaagcgcatgtttgtatgtcgagaattatcatgcgggtgactggctcgctaattctggcatggaaggatgccacatcgaattgatccggggagactaacacttgtcagaattgcaatgtgccatattccagatatcccagccggcccttctataaaccacctgcgggctcagatacctacgaagaggctcagataactcaaggacgtgcattcgaattatccctgccgcgcggaaacatcagaccaggtgcggatgctgagcgtcgagttgggtgcttgatagaccttcaccttgatctgaggttcccgtccccagagcactcgaatctccggcatcttacaggcaaaccgcaaacagtaaataatggcgagcaccatcaccatgggtacccttgcagtgccccaaaaactggctaccacctaacaattctcacgcagttttatcctctgcactttgatgtcagctttttgattcgtctgcgtacattacagcgttgagtggccagcaggaaggagaccgcggtccgagacgagtctgagggcgcgctctcgcaacttggattccggatttcttaccctgcatcgacctcggcctggagtcgatcagaaattgtcattgccagattgcctggcgaggacgggtgatatactcaaggcgttgcatcgcccacaaaacacacacttatctgcaagggagttactgcatcaggctctgctcaacagctcgtgacatcgatcgttcagctccccagcaggtgcgtgtccgcatggagcacccctcccgagacacctgcgttgggtgtcggaggagctcacatgccagggaggtgcccacattgcaccacgcgaccgcgaaataggcagacttcgggcatcctgtcatcgcatgtccgctggccgggaatcatggcctccccaccaggcgtcacgcgctgcccacctccctccccttgctgcgcagggcaccgcgttcctgtggagagccgaccacatgtccccccccaactccatgtcccccgccaccaacggctccaccaacggcgtggccatcaacggcgccaagaagctgctggacttcgacccctccgccgccccccccttcaagatcgccgacatccgcgccgccatccccccccactgctgggtgaagaacccctggcgctccctgtcctacgtgctgcgcgacctgctggtgatcctgtccttcgccgtggccgccaccaagctggactcctggaccgtgtggcccctgtactggatcgcccagggcaccatgttctgggccgtgttcgtgctgggccacgactgcggccacggctccttctccgactcctggctgctgaacaacgtgatgggccacatcctgcactcctccatcctggtgccctaccacggctggcgcatctcccacaagacccaccaccagaaccacggcaacgtggagaaggacgagtcctgggtgcccctgcccgagaaggtgtacaagtccctggacaccggcaccaagttcatgcgcttcaccatccccctgcccatgttcgcctaccccatctacctgtggcgccgctcccccggcaagaagggctcccacttcaacccctactccgacctgttcgcccccaacgagcgcacctccgtgatgatctccaccctgtgctggaccgccatggccctgctgctgtgctactcctccttcatctacggcttcctgcccgtgttcaagatctacggcgtgccctacctgatcttcgtggcctggctggacatggtgacctacctgcaccaccacggctacgagcagaagctgccctggtacaggggcaaggagtggtcctacctgcgcggcggcctgaccaccgtggaccgcgactacggcgtgatcaacaacatccaccacgacatcggcacccacgtgatccaccacctgttcccccagatgccccactaccacctggtggaggccacccaggccgccaagcacgtgctgggcaagtactaccgcgagcccaagaagtccggccccttccccttccacctgttcggctacctggtgcgctccctgggcgaggaccactacgtgtccgacaccggcgacgtggtgttctaccagtccgacccccacatccccaagttccccacctccgccaccaccaagtccaagtcctcctgagtgatccgggaggagggagtgagcggggaagggggcagccacacggggcccgtctcgacctgccacccctcccctcgtcgagccctgcccagggggcgccgcaacgagccatgcgtgtgcatgtgtctggagggcccttccaccgggcgatgtgcgagccatcctcgcctatttcaacacaccgctgccggcatgcgctccactccccccaaaaccacctcgaccctcccagggctcctcccccgccccaccctgcctgctgatatagaaaccagtgttctgtgaacgtttgacatgctcaacgagggtacaggggtgcaccaacagaggaggagtggttcacacagtcggatacactagtccttcctgtcccacaatgcttggtgaatgcagtgggttgatcaccgcggaggagctgtggcttactcgttctgatcaagggagcctctgcaccttaaccctgccaggatcgaaaccaaccttgtcagtcccgtggtgggcaacatcatcctcgtgaagctgattgaccaggaaaacatgatgagtcggtatgaggacgagcatgagtggcccaacatcgatatgacacatcttggagtttacggcaaatgtatcacacttccatcctggcttgcaccacaatattagtggacccctccttgcagtggcacggtgagaagctagtttgtagtaatcttcttaattgacgaaccagacgtgtgtaatggcctcctttgagtgatggaaggatggaacctaccccccccctccccagtactctgcggtacatccgagtaacccttccattgatcagcccaaacgcaatatgcaacgactctacatacggccaccgagtgcttattccttcgctatcaccgcacaaaaatcccatccgcgaactcatccgaggtgatagattgcgatcggggttattcgggttaaggtgcgactagggatccctgaatcttttggggatttccccgggtctcgtcctgcatgcttatcatcagtctcgtgggttatttggatcgctgcgcatgccataacagagcgctcataatatttgctgcggcggtggtgctggcaaaatcccctgcgtaccgggcgcctgtcaagccaaccccgccgtgcggcactcccctgcagatccatcaccatgatcgagcaggacggcctccacgccggctcccccgccgcctgggtggagcgcctgttcggctacgactgggcccagcagaccatcggctgctccgacgccgccgtgttccgcctgtccgcccagggccgccccgtgctgttcgtgaagaccgacctgtccggcgccctgaacgagctgcaggacgaggccgcccgcctgtcctggctggccaccaccggcgtgccctgcgccgccgtgctggacgtggtgaccgaggccggccgcgactggctgctgctgggcgaggtgcccggccaggacctgctgtcctcccacctggcccccgccgagaaggtgtccatcatggccgacgccatgcgccgcctgcacaccctggaccccgccacctgccccttcgaccaccaggccaagcaccgcatcgagcgcgcccgcacccgcatggaggccggcctggtggaccaggacgacctggacgaggagcaccagggcctggcccccgccgagctgttcgcccgcctgaaggcccgcatgcccgacggcgaggacctggtggtgacccacggcgacgcctgcctgcccaacatcatggtggagaacggccgcttctccggcttcatcgactgcggccgcctgggcgtggccgaccgctaccaggacatcgccctggccacccgcgacatcgccgaggagctgggcggcgagtgggccgaccgcttcctggtgctgtacggcatcgccgcccccgactcccagcgcatcgccttctaccgcctgctggacgagttcttctgagctgttcctaggaacgtggaggaggtgcaaggagggtgatctcaccctggtgtgtctcttcatggagctcagatcttgaaaactgtgaggtgcttatccgatacctgcttcgtgcatggcttgtgcgatatgtacacgcatttgcagattggtgggagcagcagattggtgggagcagcatagagctttagaaggggcttaggagcgggaatgtgaaactcaggcggttgggccagatgagagcgcaaagggatcccaacgctacgcaactcccttcgatggcttcaagtacggagatgtgggcatccaggattcgcatgtgctgcttcagccctcctcatgccactagcactcatttttcgactcccggattgccaggttcaagggcatcaaggagtcggagatcagccgcgccatgacctcccgctacttcgaggacctaaacgtcaatgccgaggtgcttttgcatatatttacagctaattatgatgggtgtggtgcgcgatatgcttgcaaggtctccggtgagctaatgatcggcacatcccttccgcgcatccgcaggtcgatgtcgtcattgttggggctgggtctgcgggcctctcgtgcgcctatgagctgagcaagcacccggatgtcaaggtatgggctgagcagggcacatcctcagatgatgttgctgtaattgcaattgaaacttgcggttgttcccagcacagcctcaatcaatcatgtgtgctgcgttggaaacgctatgataccccagccttcaacatggggcagggatatcgtttacacctgcttgaaccccccgcaacaggtggccatcatcgagcagggcgtcgcccctgggggtggagcgtggctggggggtcagctcttctcggctatgtgtgtgagtctaggcacggggacgggtggactgaagcaagggttgggcgcagggtgttgatatccatgtgttggacattctcgttgggaaaacaagatgtgtgtatttagtgctatctcggtggctgcattccaagctt

SEQ ID NO: 17 - DNA, 1380 bp, synthetic construct
  misc_feature 1..1380: note = Coding sequence of KASII from A. protothecoides, optimized for translation
  source 1..1380: mol_type = other DNA
atggccaccgcctccctgcccgtgcaggtggccgtgacctccacccactgcttcggcctgcgcgagccccgccgcaagcgccagtgggcccgccagacccgctgccacgcctccgccgccggcaagcccaagcgccgcgtggtggtgaccggccagggcgtggtgacctccctgggccagtccacccagcagttctacgaccagctgctggccggcgcctccggcatcacccacatcgagggcttcgacacctccgactactccaccaagatcgccggcgaggtgaagtccgtggacgccgccccctacgtggcccgcaagtgggtgaagcgcatggacgaggtgatgaagttcatgttcgtggccggcaagcaggccctggaggacgccggcctgcccttcgagggccccggcctggaggacctggaccgcaagctgtgcggcatcctgatcggcaccgccatgggcggcatgaccaccttcgcctccggcgtggaggccctgaccctgtccggccaccgcaagatgaaccccttctgcatccccttctccatcggcaacatgggcggcgccatgctggccatggacctgggcttcatgggccccaactactccatctccaccgcctgcgccaccggcaactactgcatcatctccgccgccgaccacatccgcaacggcgacgccgtgctgatgctggcgggcggcgccgacgccgccgtgatcccctccggcatcggcggcttcatcgcctgcaaggccctgtcccgccgcaacgacgcccccgagcgcgcctcccgcccctgggacgccggccgcgacggcttcgtgatgggcgagggcgccggcgtgctggtgctggaggagctggagcacgcccgcgcccgcggcgccaccatcctggccgagttcatcggcggcgcggccacctgcgacgcccaccacatgaccgagcccgagccctccggccgcggcgtgcgcctgtgcctggagcgcggcctggccgccgccggcgtggcccccgaggaggtgacctacgtgaacgcccacggcacctccacccccgccggcgacgtggccgagttccgcgccatccgcgccgtgctgggccacgacggcctgcgcatcaactcctccaagggcgccatcggccacctgctgggcgcggcgggcgccgtggaggccgtggccaccatccaggccctgcgcaccggctggctgcaccccaacctgaacctggacgagcccgacaagggcgtggacgcctccgtgctggtgggcggcgtgaaggagcaggccgacgtgaaggtggccctgtccaactccttcggcttcggcggccacaactcctgcgtgctgttccgcaagttcgaggagtga

SEQ ID NO: 18 - protein, 459 aa, synthetic construct
  REGION 1..459: note = Amino acid sequence of A. protothecoides beta-ketoacyl-ACP synthase II
MATASLPVQVAVTSTHCFGLREPRRKRQWARQTRCHASAAGKPKRRVVVTGQGVVTSLGQSTQQFYDQLLAGASGITHIEGFDTSDYSTKIAGEVKSVDAAPYVARKWVKRMDEVMKFMFVAGKQALEDAGLPFEGPGLEDLDRKLCGILIGTAMGGMTTFASGVEALTLSGHRKMNPFCIPFSIGNMGGAMLAMDLGFMGPNYSISTACATGNYCIISAADHIRNGDAVLMLAGGADAAVIPSGIGGFIACKALSRRNDAPERASRPWDAGRDGFVMGEGAGVLVLEELEHARARGATILAEFIGGAATCDAHHMTEPEPSGRGVRLCLERGLAAAGVAPEEVTYVNAHGTSTPAGDVAEFRAIRAVLGHDGLRINSSKGAIGHLLGAAGAVEAVATIQALRTGWLHPNLNLDEPDKGVDASVLVGGVKEQADVKVALSNSFGFGGHNSCVLFRKFEE

(19) United States
(12) Patent Application Publication
(10) Pub. No.: US 2024/0368646 A1
(43) Pub. Date: Nov. 7, 2024

(54) Production of Lipids and Terpenoids in Auxenochlorella Protothecoides

(71) Applicants: Phycoil Biotechnology International, Inc., Fremont, CA (US); Phycoilbiotech Korea, Inc., Seoul (KR)

(72) Inventors: Jeffrey Moseley, Fremont, CA (US); Byung-Hee Lee, Seoul (KR); Chung-Soon Im, Seoul (KR); Jane Kim, Fremont, CA (US); Dayoung Kim, Seoul (KR); Riyaz Bhat, Fremont, CA (US)

(73) Assignees: Phycoil Biotechnology International, Inc., Fremont, CA (US); Phycoilbiotech Korea, Inc., Seoul (KR)

(21) Appl. No.: 18/680,175

(22) Filed: May 31, 2024

Related U.S. Application Data
(62) Division of application No. 17/519,854, filed on Nov. 5, 2021, now Pat. No. 12,037,630.
(60) Provisional application No. 63/109,901, filed on Nov. 5, 2020.

(51) Int. Cl. C12P 7/6472; C12N 9/02; C12N 9/16; C12N 9/90; C12N 15/52

(52) U.S. Cl. CPC C12P 7/6472; C12N 9/0071; C12N 9/0083; C12N 9/16; C12N 9/90; C12N 15/52; C12Y 114/19; C12Y 114/19002; C12Y 301/02014; C12Y 505/01018

(57) ABSTRACT

Methods to produce oils with modified profiles of fatty acid, carotenoids and/or terpenoids in microalgal mutants are provided. Microalgal mutants produce the oil containing fatty acids, carotenoids and/or terpenoids of a modified profile with a disruption or ablation of one or more alleles of an endogenous polynucleotide or comprising an exogeneous gene are also provided.

### CROSS-REFERENCE TO RELATED APPLICATION

This application claims the benefit of U.S. Provisional Application No. 63/109,901 filed on Nov. 5, 2020, the disclosures of which are herein incorporated by reference in their entirety.

### TECHNICAL FIELD

The present invention describes microalgae having a functional oil-producing ability comprising an altered profile of fatty acids, carotenoids and/or terpenoids and a method of extracting oil using the microalgae. More specifically, it relates to a method for producing oil having modified fatty acids, carotenoids and/or terpenoids content compared to wild-type microalgae, such as Auxenochlorella protothecoides with inhouse strain designation as PB5.

### BACKGROUND ART

Industrial production of lipids and fats from oleaginous microorganisms has been investigated since the late 19th century, but the promise of these “single cell oils” has only been realized in the last 20-30 years¹. Oleaginous microorganisms trigger biosynthesis of storage lipids in response to imbalances between carbon (C) supply and other major nutrients such as nitrogen (N) or phosphorus (P) that are required for growth. This response allows accumulation of excess C under conditions where limitation for other nutrients prevents cell growth and division. Microbial storage lipids are largely composed of triacylglycerides (TAGs), which can be mobilized and used for rapid growth when the limiting nutrient or nutrients become available. Subsets of yeasts, fungi, and algae, but very few bacteria are oleaginous¹. In the case of some algae, oil production can be stimulated by photosynthesis or by heterotrophic fermentation of sugars, but there is little economic incentive to make lipids which have similar composition or properties to commodity plant oils. In the early 2000s there was a flurry of interest and investment in photosynthetic production of biofuels from algae, but this has now largely faded due to the unfavorable energy return and economics of fuel production from aquatic microbes^{2,3}. Heterotrophic oil productivity is substantially higher than is the case for photoautotrophic production, but the price of common sugar feedstocks is seldom any less that one quarter of the price of typical commodity oils, and the lipid yield for most heterotrophic processes is less than 25%. The economics can improve if the carbon source comes from a low value waste stream (e.g., lactose from cheese production, cellulosic sugars from agricultural waste).

On the other hand, heterotrophic production of very long chain polyunsaturated fatty acids (VLCPUFAs) from algae, thraustochytrids and yeasts provides a roadmap for successful commercialization of microbial oils. Docosahexaenoic acid (DHA) from the dinoflagellate algae Crypthecodinium cohnii and arachidonic acid (ARA) from the yeast Mortierella alpina are both important components of infant formula¹. These fatty acids are important components of human breast milk and are vital for brain and nervous system development⁴. The microbial oils address a particular market need for which there are no appropriate substitutes, since the only other significant sources of VLCPUFAs come from fish oils which are subject to contamination with toxins and heavy metals.

Other successful commodity products from aquatic microalgae follow the VLCPUFA model. Astaxanthin and beta-carotene are key carotenoids for human nutrition; the former is one of the most powerful antioxidants known, while beta-carotene also has strong antioxidant activity and is a pro-vitamin A supporting vision⁵. Synthetic astaxanthin derived from petrochemicals is used extensively as a colorant in fish farming but is not approved for human consumption in food or supplements. The major natural source of astaxanthin is the chlorophyte Haematococcus lacustris (also H. pluvalis), which is grown photoautotrophically in ponds or bioreactors. Haematococcus cultures can produce up to 5% of their biomass as astaxanthin when they are subjected to nutrient and high light stress. Astaxanthin is also produced commercially by the heterotrophic fermentation of the yeast Phaffia rhodozyma, but the carotenoid content of the biomass is much lower than Haematococcus^{6,7}. Natural beta-carotene is extracted from another chlorophyte, the halophile Dunaliella salina, which is grown photoautotrophically in large salt ponds. Both Haematococcus and Dunaliella fetch high prices for the biomass, but photoautotrophic cell densities are too low and the production costs for harvesting and extraction are too high for the natural sources to reach the scale where they can compete with synthetic carotenoids.

Squalene is an important component of human sebum (12%), a fact that justifies its role in the physiology of skin⁸, its action in skin hydration, repairing of the damaged skin, and rejuvenating the aging skin was demonstrated. The emollient and hydration properties of squalene and its biocompatibility with skin make squalene an important component in cosmetic formulations (moisturizing creams, makeup, lipstick, and nail and hair products).

Furthermore, squalene appears to play an essential role in protecting skin from free radical oxidative damage. Squalene acts in skin as a quencher of singlet oxygen, protecting by this mechanism the skin surface from lipid peroxidation due to exposure to UV light.

Squalene has been known to play diverse biological roles as an antioxidant^{9,10}, anti-cancer agent^{10,11}, age defyer^{12,13}, chemo preventive agent^{14,15,16}, antibacterial agent^{13,17}, adjuvant for vaccines and drug carrier^{18,19}, and detoxifier^{11,20} among others.

Squalene has proved to be a well-tolerated, non-toxic cytoprotective agent that mitigates undesirable side effects of cancer chemotherapy. Many anticancer therapies damage normal healthy tissues, even to the point of organ toxicity. These toxic secondary effects can limit the anticancer drug dosage, and even lead to treatment failure. Squalene is effective at scavenging and detoxifying free radicals produced by chemotherapeutic or radiation therapy agents.

The role of squalene is not just confined to these applications, but it is also a precursor to thousands of bioactive molecules, including steroids and hopanoids. Consequently, many chemicals, food, cosmetic, and pharmaceutical industries have started to use squalene extensively. It also acts as a boosting agent, or adjuvant, that improves the immune system and makes vaccines more effective. Over the last decade, global squalene demand has increased and gained much public and scientific attention. In 2014, the global squalene market demand was about 2.67 kilotons²¹, with a projected value of 241.9 million USD by 2022, with major revenues expected from the personal care and cosmetic products²². In order to fulfill this ever-increasing demand of squalene, a pressing need has arisen to produce squalene in a renewable and sustainable manner.

Squalene is harvested from deep-sea sharks and exists in high concentration in shark liver. However, the intensive fishing of these sharks puts in danger the existence of these species, with many of them being close to extinction as their reproductive cycle is quite long and the growth is slow.

Most plant seed oils contain small amounts of squalene²³. This phytosqualene has superior qualities compared to shark squalene, in that it is highly stable, generally free of heavy metal contamination, odorless, and colorless. In spite of containing significant amounts of squalene, plants oils are not ideal sources. Some plants are strictly seasonal, and squalene content varies greatly geographically. Oilseed crops require the appropriate climatic conditions, soil quality, scheduled irrigation or sufficient rainfall, fertilizer, and pest management. The process of cultivation is labor intensive and correspondingly the amount of squalene produced from plant sources is not sufficient to fulfill the increasing worldwide demand.

Several microalgae and other microorganisms accumulate comparable levels of squalene to plant oils. Short generation times and ease of genetic engineering make microalgae a better alternative for squalene production than plants.

Native biosynthetic pathways which include squalene as an intermediate are present in many microalgae, and these pathways can be transformed or extended to convert the organisms into “cellular factories” for squalene production.

Auxenochlorella protothecoides is one of the most oleaginous species of microalgae and can be cultivated heterotrophically with productivity in commercial scale²⁴. Metabolic engineering using genetic tools on this organism has strong potential to produce variety products including carotenoids, terpenoids, and other active ingredients, which can be an important alternative source of currently depleted natural resources.

### BRIEF SUMMARY OF THE INVENTION

The inventors of the present application have developed the Trebouxiophyte alga, Auxenochlorella protothecoides PB5, as a biotechnology platform for the heterotrophic production of valuable lipids, carotenoids, terpenoids, and other compounds. Efficient transformation and facile gene targeting by homologous recombination are features of this system. Targeted knockouts and knock-ins of fatty acid and lipid biosynthetic pathway genes enabled the improved synthesis of polyunsaturated fatty acids. Valuable ketocarotenoids were produced by disrupting endogenous carotenoid biosynthetic genes and expressing heterologous beta-carotene ketolase transgenes. Squalene accumulation was increased by blocking phytosterol biosynthesis. A. protothecoides PB5 can be employed as a general platform for the photoautotrophic, mixotrophic, or heterotrophic production of valuable biomolecules. Here the inventors of the present application demonstrate the utility of the system for the biosynthesis of modified fatty acids, lipids, carotenoids, and other terpenoids (FIG. 1).

It is an object of the present invention to provide mutant microalgae having functional oil-producing ability comprising modified profiles of fatty acids, carotenoids and/or terpenoids as proposed to solve the above problems.

Another object of the present invention is to provide a method for producing oil using the microalgal mutant.

Another object of the present invention is to provide oil prepared by the above production method.

### ADVANTAGEOUS EFFECTS

The present application shows that when using a microalgal mutant in which Auxenochlorella protothecoides PB5 microalga genes are knocked out or knocked in by homologous recombination, an oil containing fatty acids, carotenoids and/or terpenoids of an altered profile compared to that of wild type thereof can be effectively extracted.

### DESCRIPTION OF THE DRAWING

FIG. 1 shows a diagram of the Isoprenoid pathway, an essential metabolic pathway present in microalgae.

FIGS. 2A-B show the sequence of the transforming DNA from the LCYE-1 disruption construct pPB0014. (SEQ ID NO: 1)

FIGS. 3A-C show the sequence of the transforming DNA from the LCYE-2 disruption construct pPB0038. (SEQ ID NO: 2)

FIG. 4 shows spectrophotometric and HPLC analysis of carotenoid pigments from wild-type A. protothecoides PB5, 14-2, and 38-25. Absorbance spectra of acetone-methanol-extracted pigments from wild-type A. protothecoides PB5, LCYE-1 disruption strain 14-2, and LCYE-1/LCYE-2 double knockout strain 38-25. Peak absorbances are indicated for each curve (A). HPLC chromatograms showing carotenoid pigments present in oil extracted from the same strains as in A (B).

FIG. 5 shows the sequence of the expression module cloned into the KpnI site of pPB0038 to generate construct pPB0120. (SEQ ID NO: 3)

FIG. 6. Nucleotide sequence of the ApSAD2tp_CrBKT1 expression module from pPB0123. (SEQ ID NO: 4)

FIG. 7A shows the amino acid sequence of native CrBKT1. The predicted plastid transit peptide is underlined. (SEQ ID NO:5)

FIG. 7B shows the amino acid sequence of chimeric ApSAD2tp_CrBKT1. The predicted ApSAD2 plastid transit peptide is underlined. (SEQ ID NO:6)

FIG. 8 shows shake flask cultures illustrating the color differences due to engineered alterations in the carotenoid profiles (A). HPLC chromatograms showing carotenoid pigments present in oil extracted from the same strains as in A (B).

FIGS. 9A-C show the sequence of the transforming construct pPB0065, targeting disruption of ApSQE-2. (SEQ ID NO: 7)

FIGS. 10A-B show the sequence of the transforming DNA from pPB0077. (SEQ ID NO: 8)

FIG. 11 shows the chromatograms of HPLC analysis of the standard squalene (A), the accumulation of squalene in SQE double knockout strain 77B-21 (B).

FIG. 12 shows the amino acid sequence of A. protothecoides 1-deoxy-D-xylulose 5-phosphate synthase (DXS). The predicted plastid transit peptide is underlined. (SEQ ID NO:9)

FIG. 13 shows the amino acid sequence of A. protothecoides 1-deoxy-D-xylulose 5-phosphate reductoisomerase (DXR). (SEQ ID NO:10)

FIG. 14 shows the amino acid sequence of A. protothecoides farnesyl diphosphate synthase (FDPS). (SEQ ID NO:11)

FIG. 15 shows the amino acid sequence of A. protothecoides squalene synthase (SQS). (SEQ ID NO:12)

FIGS. 16A-C show the sequence of the transforming construct pPB0039, targeting insertion of the ApSAD2 promoter upstream of the FAD3-1 coding sequence. (SEQ ID NO: 13)

FIG. 17 shows the nucleotide sequence of the ApFATA promoter in pPB0041 SEQ ID NO: 14)

FIGS. 18A-C show the sequence of the transforming construct pPB0118, targeting AtPDCT and ScSUC2 expression to the THI4 locus. (SEQ ID NO: 15)

FIGS. 19A-C show the sequence of the transforming construct pPB0142, targeting LuFAD3A and neoR expression to the THI4 locus. (SEQ ID NO: 16)

FIG. 20 shows the growth of strains targeting one or both alleles of THI4. Wild-type A. protothecoides PB5 is unable to grow without added thiamine. Thiamine prototrophy is observed in strain 118B-8, expressing AtTHIC, targeted to one allele of THI4. Disruption of the second THI4 allele by pPB0142 renders the transformants thiazole auxotrophs.

FIG. 21 shows the coding sequence of KASII from A. protothecoides, optimized for translation. (SEQ ID NO:17)

FIG. 22 shows the amino acid sequence of A. protothecoides beta-ketoacyl-ACP synthase II. (SEQ ID NO:18)

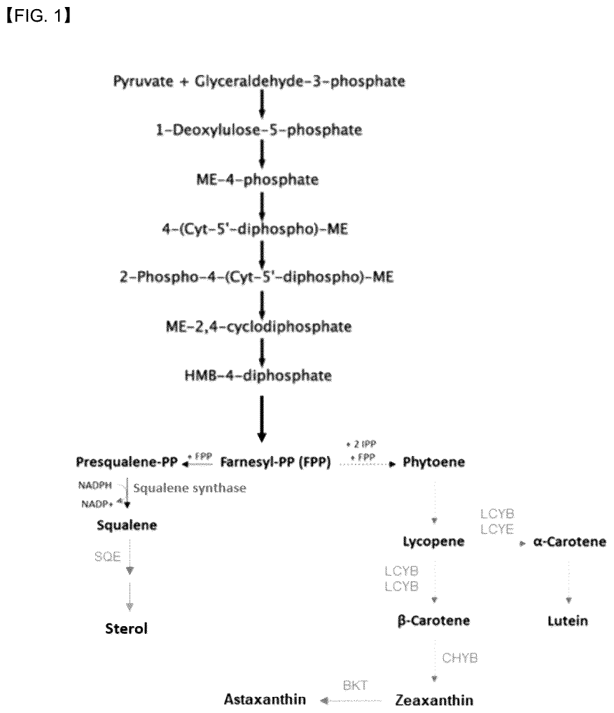

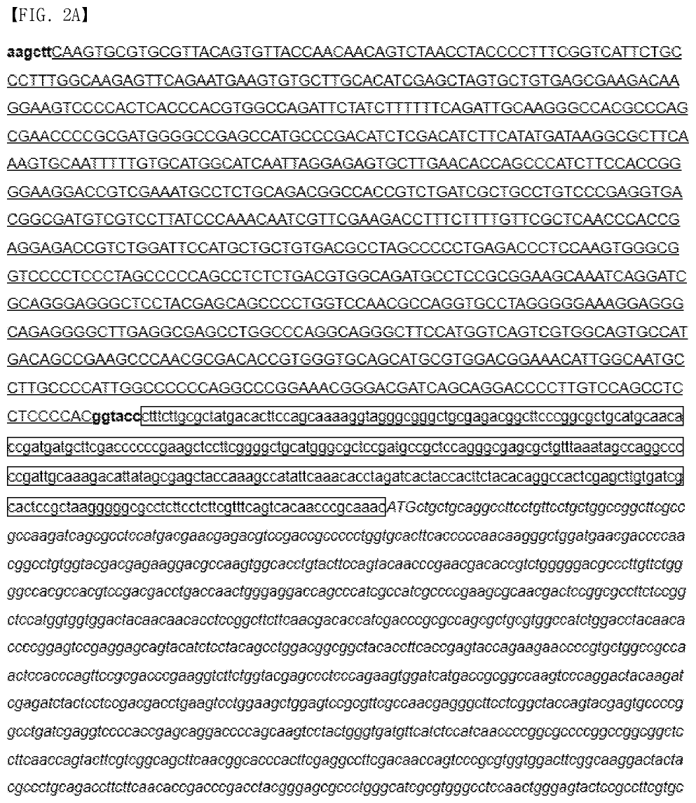

### DETAILED DESCRIPTION OF THE INVENTION

Unless otherwise defined herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner like a term “comprising.” The transitional terms/phrases (and any grammatical variations thereof) “comprising,” “comprises,” “comprise,” “consisting essentially of,” “consists essentially of,” “consisting,” and “consists of” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the value as determined by one of ordinary skills in the art, which will depend in part on how the value is measured or determined, i.e., the limitations of the measurement system. Where values are described in the application and claims unless otherwise stated the term “about” meaning within an acceptable error range for the particular value should be assumed. In the context of compositions containing amounts of ingredients where the term “about” is used, these compositions contain the stated amount of the ingredient with a variation (error range) of 0-10% around the value (X±10%).

An “allele” refers to a version of a gene at the same place on homologous chromosomes. An allele may encode the same or similar protein.

“Exogenous gene” shall mean a nucleic acid that codes for the expression of an RNA and/or protein that has been introduced into a cell (e.g., by transformation/transfection), and is also referred to as a “transgene”. A cell comprising an exogenous gene may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

“Fixed carbon source” is a molecule(s) containing carbon, typically an organic molecule that is present at ambient temperature and pressure in solid or liquid form in a culture media that can be utilized by a microorganism cultured therein. Accordingly, carbon dioxide is not a fixed carbon source.

“Microalgae” are eukaryotic microbial organisms that contain a chloroplast or other plastid, and optionally that can perform photosynthesis, or a prokaryotic microbial organism capable of performing photosynthesis. Microalgae include obligate photoautotrophs, which cannot metabolize a fixed carbon source as energy, as well as heterotrophs, which can live solely off a fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis.

In connection with a recombinant cell, the term “knockdown” refers to a gene that has been partially suppressed (e.g., by about 1-95%) in terms of the production or activity of a protein encoded by the gene.

Also, in connection with a recombinant cell, the term “knockout” refers to a gene that has been completely or nearly completely (e.g., >95%) suppressed in terms of the production or activity of a protein encoded by the gene. Knockouts can be prepared by ablating the gene by homologous recombination of a nucleic acid sequence into a coding sequence, gene deletion, mutation, or other methods. When homologous recombination is performed, the nucleic acid that is inserted (“knocked-in”) can be a sequence that encodes an exogenous gene of interest or a sequence that does not encode for a gene of interest.

An “oleaginous” cell is a cell capable of producing at least 20% lipid by dry cell weight, naturally or through recombinant or classical strain improvement. An “oleaginous microbe” or “oleaginous microorganism” is a microbe, including a microalga that is oleaginous (especially eukaryotic microalgae that store lipid). An oleaginous cell also encompasses a cell that has had some or all its lipid or other content removed, and both live and dead cells.

In connection with a functional oil, a “profile” is the distribution of species or triglycerides or fatty acyl groups within the oil. A “fatty acid profile” is the distribution of fatty acyl groups in the triglycerides of the oil without reference to the attachment to a glycerol backbone. Fatty acid profiles are typically determined by conversion to a fatty acid methyl ester (FAME), followed by gas chromatography (GC) analysis with flame ionization detection (FID). The fatty acid profile can be expressed as one or more percent of fatty acid in the total fatty acid signal determined from the area under the curve for that fatty acid.

“Recombinant” is a cell, nucleic acid, protein, or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell. Recombinant cells can, without limitation, include recombinant nucleic acids that encode for a gene product or for suppression elements such as mutations, knockouts, antisense, interfering RNA (RNAi), or dsRNA that reduce the levels of the active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid.

The terms “triglyceride”, “triacyl glyceride” and “TAG” are used interchangeably as is known in the art.

In the present disclosure, ranges are stated in shorthand, to avoid having to set out at length and describe each value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. When ranges are used herein, combinations and sub-combinations of ranges (e.g., subranges within the disclosed range), specific embodiments therein are intended to be explicitly included.

According to one aspect of the present invention, a method for producing oil comprising a fatty acid, carotenoid, and/or terpenoid of a modified profile using a gene knockout or knock-in microalgal mutant by homologous recombination is provided.

According to one aspect of the present invention, a method for producing oil, characterized in that the weight ratio of zeaxanthin to lutein is high compared to the oil produced from wild-type microalgae, is provided.

According to one aspect of the present invention, a method for producing an oil comprising astaxanthin is provided.

According to one aspect of the present invention, a method for producing an oil comprising squalene is provided.

According to one aspect of the present invention, a method for producing a functional oil, characterized in that the weight ratio of omega-6 to omega-3 is low compared to the oil produced from wild-type microalgae, is provided. Preferably, the ratio of the oil ranges between 1:1 to 5:1 compared to the oil produced from the wild type microalgae which is 7:1.

According to one aspect of the present invention, a method for producing a functional oil, characterized in that the omega-3 fatty acids increased 3-5-fold and the overall PUFA increased 2-3-fold compared to the wild-type strain.

According to one aspect of the present invention, the microalgal mutant may be a gene knockout or knock-in microalgal mutant by homologous recombination. Preferably, the microalgae may be Auxenochlorella genus, more preferably, Auxenochlorella protothecoidesPB5.

According to one aspect of the present invention, the microalgae mutant is to provide a method for producing functional oil, characterized in that one or more of the alleles LCYE-1 and LCYE-2 of the Lycopene Cyclase Epsilon gene are knocked out.

According to one aspect of the present invention, the microalgal mutant strain may be to provide a method for producing a functional oil, characterized in that one or more of the alleles SQE-1 and SQE-2 of the Squalene Epoxidase gene are knocked out.

According to one aspect of the present invention, the microalgal mutant may be to provide a method for producing a functional oil, characterized in that the native FAD3 promoter is replaced with a Stearoyl-ACP Desaturase (SAD2) promoter, or the native FAD3 promoter is replaced with an acyl-ACP thioesterase (FATA) promoter.

According to one aspect of the present invention, it may be to provide the use of microalgae as a platform to produce functional oils comprising fatty acids, carotenoids, and/or terpenoids of altered profile by modification of the isoprenoid pathway.

According to an aspect of the present invention, it may be to provide a mutant microalga for the production of oil containing fatty acids, carotenoids, and/or terpenoids of an altered profile.

According to an aspect of the present invention, it may be to provide an oleaginous microalgae mutant that produce a functional oil, the microalgae comprising an ablation of one or more alleles of an endogenous polynucleotide or comprising an exogenous gene.

According to one aspect of the present invention, the microalgal mutant is a microalgal mutant knocked out or knocked-in by homologous recombination, preferably, at least one of the alleles LCYE-1 and LCYE-2 of lycopene cyclase epsilon characterized by knocking out, or knocking out at least one of alleles SQE-1 and SQE-2 of squalene epoxidase, or replacing the native FAD3 promoter with a Stearoyl-ACP Desaturase (SAD2) promoter or a mutant strain in which the native FAD3 promoter is replaced with the promoter of the A. protothecoides FATA gene encoding acyl-ACP thioesterase.

Hereinafter, the present invention will be explained in detail.

As an aspect for achieving the object of the present invention, the present invention provides an Auxenochlorella protothecoides mutant for producing a fatty acid, a carotenoid, or terpenoid having a modified profile.

As an embodiment, the Auxenochlorella protothecoides mutant may be a mutant of A. protothecoides PB5.

The Auxenochlorella protothecoides mutant of the present invention may be prepared using general mutation treatment methods.

In the present invention, “mutation” refers to a change in a nucleotide sequence due to the insertion, deletion, or substitution of a base into the original nucleotide sequence. As a means of mutation, the number of inserted bases may be different depending on the mutation and thus is not limited thereto. “Deletion mutation” means a mutation in which a base is removed from the original nucleotide sequence, and “substitution mutation” means that an original nucleotide is changed to another base without changing the number in the original nucleotide sequence.

In a specific embodiment of the present invention, pPB0014 has a transforming DNA with the nucleotide sequence of SEQ ID NO: 1 which is a DNA construct in which LCYE-1 encoding one allele of Lycopene Cyclase Epsilon in Auxenochlorella protothecoides PB5 is prepared in order to change the ratio of lutein derived from alpha-carotene and zeaxanthin derived from beta-carotene compared to wild type. Then, strain 14-2 was prepared by introducing pPB0014. Afterward, in order to generate a double knockout strain completely lacking lutein, a carotenoid derived from alpha-carotene, pPB0038, a DNA construct having a transforming DNA with the nucleotide sequence of SEQ ID NO: 2, in which LCYE-2 encoding the second allele of Lycopene Cyclase Epsilon was prepared. The mutant strain 38-25 is prepared by transforming the strain 14-2 with said pPB0038.

It was confirmed that the above strains increased the ratio of zeaxanthin to lutein compared to the wild type.

In a further embodiment, to prepare a keto-carotenoid, an expression module for C. reinhardtii beta-carotene ketolase1 (CrBKT1) SEQ ID NO: 4, was introduced into the pPB0038 backbone to produce construct pPB0123, which targeted the LCYE-2 single allele. Alternatively, strains in which LCYE alleles were disrupted were prepared. In addition, when a single LCYE allele is knocked out, 4-keto-lutein and astaxanthin are produced, while when both LCYE alleles are knocked out strains almost exclusively produced astaxanthin.

In a specific embodiment of the present invention, to obtain squalene, ApSQE-2 encoding the Squalene Epoxidase allele 2 in Auxenochlorella protothecoides PB5 is knocked out. DNA construct having a transforming DNA with the nucleotide sequence of SEQ ID NO: 7 pPB0065 was prepared and introduced into wild-type Auxenochlorella protothecoides PB5 to prepare strain 65-4. Then, in order to generate a double knockout strain, pPB0077, a DNA construct having nucleotide sequence SEQ ID NO: 8 was constructed and transformed into the strain 65-4 in which SQE-1 encoding Squalene Epoxidase allele 1 was knocked out. The prepared mutant strain was named strain 77B-21.

It was confirmed that the above strains can accumulate squalene while the wild type cannot.

In a specific embodiment of the present invention, the native FAD3 promoter in A. protothecoides PB5 is replaced with a promoter of a gene that is strongly up-regulated during lipid production, thereby activating the endogenous FAD3 gene encoding Fatty acid Desaturase 3 to reduce the ratio of omega-6 to omega-3 compared to the wild type. Specifically, construct pPB0039 having a transforming DNA with the nucleotide sequence of SEQ ID NO: 13 was constructed in which the ApSAD2 promoter was inserted upstream of the FAD3-1 coding sequence. Alternatively, construct pPB0041 was constructed, in which the native FAD3 promoter was replaced with the promoter of the FATA gene encoding acyl-ACP thioesterase SEQ ID NO: 14. Plasmid construct pPB0039 and pPB0041 were introduced into wild-type A. protothecoides PB5 to obtain strains 39-1 and 39-9 strains 41-1 and 41-3, respectively. It was confirmed that the strains increased the ratio of omega 3 to omega 6 compared to the wild type.

In a specific embodiment of the present invention, transfer of C18:1 between DAG and phospholipids was improved by transforming a construct, pPB0118 SEQ ID NO:15, encoding Arabidopsis phosphatidylchloline:diacylglycerol choline phosphotransferase (PDCT) into strain 41-3. As a result, C18:2 accumulation increased by 2.5-fold in strains 118B-8 and 118B-20, indicating that expression of AtPDCT during lipid production caused significant enhancement of FAD2 activity, and the incorporation of C18:2 into TAG was favored over desaturation by FAD3.

In a specific embodiment of the present invention, the accumulation of fatty acids C18:3 (ALA) was increased by the introduction of a construct, pPB0142 SEQ ID: NO:16, encoding heterologous (Linum usitatissimum) FAD3A into strain 118B-8. As a result, ALA accumulation increased about 3% in strain 142B-11.

Auxenochlorella protothecoides PB5 is a superior system for generating engineered microalgae strains due to its ease of transformation and facile homologous recombination that does not require riboprotein-mediated gene editing. PB5 has a higher intrinsic capacity than non-photosynthetic heterotrophic platforms for production of carotenoids and other terpenoids due to the high flux through these biosynthetic pathways during photosynthetic growth.

The Mutants of the present invention are more industrially useful in that they may provide oils having fatty acids, carotenoids, and squalene content of a profile different from that produced in wild-type Auxenochlorella protothecoides.

In some cases, the percent (w/w) of zeaxanthin in the carotenoids produced by using the microalgal mutants of the present invention is 2-3-fold higher compared to the wild-type microalgae and the zeaxanthin is present as a major carotenoid. In a specific embodiment, the percent of zeaxanthin produced ranges between 40 to 90% (w/w) of the total identified carotenoids.

In some cases, the oil produced may contain keto carotenoids, such as the mixture of keto lutein, and astaxanthin, and the astaxanthin is present as a major carotenoid. In a specific embodiment, the percent of keto carotenoids produced ranges between 20-90% (w/w) of the total identified carotenoids.

In some cases, the oil produced may contain squalene. In a specific embodiment, the amount of squalene in the oil produced ranges from 300 to 1300 ppm.

In some cases, the weight ratio of omega-6 to omega-3 in the oil produced by using the microalgal mutants of the present invention is low compared to the oil produced from wild-type microalgae which are 7:1. In a specific embodiment the percent ratio of omega-6 to omega-3 in the oil produced ranges from 1:1 to 5:1.

The mutant of the present invention may grow appropriately in a growth environment (light conditions, temperature conditions, medium, etc.) capable of culturing conventional Auxenochlorella protothecoides.

The mutant of the present invention may be cultured according to the culture conditions of general Auxenochlorella protothecoides, and specifically, a culture medium capable of culturing algae under weak light conditions may be used. To culture a specific microorganism, it may include a nutrient material required for a culture target, that is, a microorganism to be cultured, and maybe mixed by adding material for a special purpose. The medium includes an all-natural medium, synthetic medium, or selective medium. The Auxenochlorella protothecoides mutant may be cultured according to a conventional culture method.

The pH of the culture medium is not particularly limited if the Auxenochlorella protothecoides may survive and grow, for example, it is viable at pH 5 or higher, specifically at pH 6 to 8.

The Auxenochlorella protothecoides mutant of the present invention may produce modified profiles of fatty acids, carotenoids, and terpenoids in cells, so that oils extracted from mutants of the present invention may be effectively used as raw materials for pharmaceuticals, cosmetics, food, feed, etc.

In this aspect, the present invention provides a composition comprising the oil derived from the Auxenochlorella protothecoides mutant. The composition may be a cosmetic composition, a food composition, a composition for a food additive, a feed composition, a composition for a feed additive, a pharmaceutical composition, a raw material composition for food, a raw material composition for feed, a raw material composition for pharmaceutics or a raw material composition for cosmetics.

The composition may be used as a raw material for food, feed or pharmaceutics, and may be used as a formulation for oral administration or parenteral administration. For example, it may be used as a formulation for oral, transdermal or injection administration. Accordingly, the composition of the present invention may be a composition for oral administration in that the composition may be orally supplied to be included in food, medicine, or feed.

In the case of compositions for oral administration may be formulated as powders, granules, tablets, pills, dragees, capsules, liquids, gels, syrups, slurries, suspensions, etc. by using methods known in the art. For example, oral preparations may be obtained by mixing the active ingredient with excipients, grinding the mixture, adding suitable additives, and processing it into a granule mixture to obtain tablets or sugar tablets. Examples of suitable excipients include sugars, including lactose, dextrose, sucrose, sorbitol, mannitol, xylitol, erythritol and maltitol, and starches, including corn starch, wheat starch, rice starch and potato starch, cellulose, including methylcellulose, sodium carboxymethylcellulose, and hydroxypropylmethyl-cellulose, and the like, fillers such as gelatin, polyvinylpyrrolidone, and the like may be included. In addition, cross-linked polyvinylpyrrolidone, agar, alginic acid, or sodium alginate may be added as a disintegrant if necessary.

The composition may be used for human and animal health promotion. Specifically, the mutant of the present invention has oil production ability with enhanced antioxidant pigment content, so it is not easily oxidized, and functionally, it is possible to provide an oil superior to conventional microalgae-derived vegetable oil in antioxidant activity, and it can be effectively used as a raw material for health functional food, feed, or medicine.

In addition, since the composition may be added to food or feed to achieve a special purpose use, in this respect it may be a food composition, a composition for food additives, a feed composition or a composition for feed additives. When the composition is used in feed or food, it is possible to maintain or enhance body health by pigments and lipids including zeaxanthin produced by the mutant and accumulated in cells.

In the present invention, “additive” is included as long as it is a material added to food or feed other than the main raw material, and specifically, it may be an effective active material having functionality in food or feed.

In the present invention, the composition for feed may be prepared in the form of fermented feed, compounded feed, pellet form, and silage. The fermented feed may include a functional oil derived from the mutant of the present invention, and additionally include various microorganisms or enzymes.

The composition is mixed with a carrier commonly used in the food or pharmaceutical field, such as tablets, troches, capsules, elixirs, syrups, powders It can be prepared and administered in the form of powder, suspension, or granules. As the carrier, binders, lubricants, disintegrants, excipients, solubilizers, dispersants, stabilizers, suspending agents, and the like may be used. The administration method may be an oral, or parenteral method, but preferably oral administration. In addition, the dosage may be appropriately selected according to the absorption of the active ingredient in the body, the inactivation rate and excretion rate, the age, sex, condition of the subject, and the like. The pH of the composition can be easily changed according to the manufacturing conditions of the drug, food, cosmetics, etc. in which the composition is used.

The composition may include 0.001 to 99.99% by weight, preferably 0.1 to 99% by weight of any one selected from the group consisting of the microalgal mutants of the present invention, the culture of the mutants, the dried product of the mutant, or the culture thereof, and the extract of the mutant or the culture thereof, and the functional oil derived from the mutant, based on the total weight of the composition, and the method of using the composition and the content of the active ingredient may be appropriately adjusted according to the purpose of use.

The mutant may be included in the composition in its own or dried form, and the culture of the mutant may be included in the composition in a concentrated or dried form. In addition, the dried product refers to the dried form of the mutant or its culture and maybe in the form of a powder prepared by freeze-drying or the like.

In addition, the extract means that obtained by extraction from the mutant of the present invention, its culture medium or its dried product, an extract using a solvent, etc. Thus, the mutant of the present invention includes those obtained by crushing the mutant of the present invention. Specifically, the oil with the modified profile accumulated in the cells of the mutant of the present invention may be extracted and separated by a physical or chemical method.

In addition, the method for producing oil with the modified profile according to the present invention may include culturing the mutant of the present invention. In addition, the production method may further include; after the culturing step, isolating the mutant of the present invention from the culture.

The culture may be performed in a medium of pH 5.0 to 8.0 conditions. In addition, it may be carried out under a weak light condition, specifically, a light intensity condition in the range of 0.1-1, 1-3, or 3-5 μmol photons/m² s.

The production method may further include, in addition to the culturing step, a concentration step to increase the content of algae after culturing, and a drying step of drying by further reducing the moisture of the algae that has undergone the concentration step. However, the concentration step or the drying step is not necessarily required, and in general, the concentration and drying method commonly used in the field to which the present invention pertains, and it can be carried out using a machine.

The production method may further include the step of purifying the material isolated from the culture, which may be performed by a conventional purification method in the art to which the present invention pertains.

### DETAILED DESCRIPTION OF THE EMBODIMENTS

Hereinafter, the present invention will be explained in detail through Examples and Experimental Examples, but these Examples and Experimental Examples are presented only as of the illustration of the present invention, and the scope of the present invention is not limited thereby.

The oleaginous Trebouxiophyte alga, Auxenochlorella protothecoides, stores copious amounts of triacylglyceride oil under conditions where the nutritional carbon supply is in excess, but cell division is inhibited due to the limitation of other essential nutrients. Heterotrophically grown Auxenochlorella strains also degrade chlorophyll and down-regulate photosynthesis but maintain significant levels of the yellow carotenoids lutein and zeaxanthin. Bulk biosynthesis of fatty acids with carbon chain lengths up to C18 occurs in the plastids; fatty acids are then exported to the endoplasmic reticulum where incorporation into triacylglycerides (TAGs) occurs. Lipids are stored in large cytoplasmic organelles called lipid bodies until environmental conditions change to favor growth, whereupon they are rapidly mobilized to provide energy and carbon molecules for anabolic metabolism. Wild-type A. protothecoides storage lipid is comprised mainly of oleic (˜68%), palmitic (˜12%), and linoleic (˜13%) acids, with minor amounts of stearic, myristic, α-linolenic, and palmitoleic acids. This fatty acid profile results from the relative activities and substrate affinities of the enzymes of the endogenous fatty acid biosynthetic pathway. A. protothecoides is amenable to manipulation of fatty acid and lipid biosynthesis using molecular genetic tools, enabling the production of oils with fatty acid profiles that are very different from the wild-type composition. Similarly, the carotenoid and phytosterol profile of the lipid fraction can be altered by genetic engineering of terpenoid biosynthesis pathways.

We have demonstrated efficient transformation and facile nuclear gene targeting via homologous recombination in A. protothecoides, PB5. In the following examples, we leverage our ability to perform gene knockouts and knock-ins to produce algal oils with modified fatty acid, carotenoid, and terpenoid profiles.

Wild type A. protothecoides PB5 was obtained from the University of Texas Culture Collection of Algae (UTEX catalog number 250), and it is available to the public to purchase via webpage www.utex.org.

### EXAMPLE 1. PRODUCTION OF STRAINS WITH ALTERED CAROTENOID PROFILES

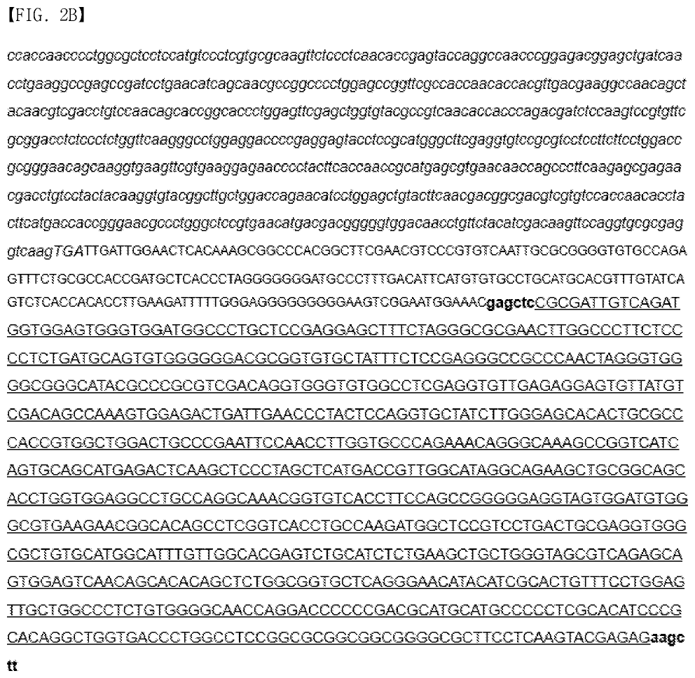

Lutein is the predominant carotenoid that accumulates in heterotrophic A. protothecoidescells. To alter the ratio of lutein (derived from alpha-carotene) to zeaxanthin (derived from beta-carotene) we made a DNA construct to disrupt LCYE-1, encoding one allele of lycopene cyclase epsilon in A. protothecoides PB5. In general, flanking regions, promoters, and terminator sequences were PCR amplified from PB5 genomic DNA, and codon-optimized synthetic genes were amplified from plasmid DNA. Herculase II Fusion Enzyme (Agilent, USA) was used for PCR amplification. 50 ml amplification reactions contained 100-500 ng genomic DNA template, 250 micromolar dNTPs, 0.25 micromolar primers, and 0.5 ml Herculase II fusion DNA polymerase. Initial denaturation was at 95° C. for 2 minutes or at 98° C. for 4 minutes for highly GC-enriched templates. PCR products were amplified using 30-35 cycles of 95° C. or 98° C., 20 seconds, 55-65° C. annealing for 20 seconds, and extension at 72° C. for 30 seconds per kb. The sequence of the transforming DNA from the LCYE-1 disruption construct pPB0014 is shown below in FIG. 2.

Relevant restriction sites used to generate linear DNA and for cloning are indicated in lowercase, bold, and are from 5′-3′ HindIII, KpnI, SaI, and HindIII. Underlined sequences at the 5′ and 3′ flanks of the construct represent genomic DNA from A. protothecoides PB5 that enable targeted integration of the transforming DNA via homologous recombination at the LCYE-1 locus. Proceeding in the 5′ to 3′ direction, the Chlamydomonas reinhardtii TUB2 promoter (CrTUB2) driving the expression of the Saccharomyces cerevisiae SUC2 gene (ScSUC2, codon-optimized for expression in A. protothecoides and encoding sucrose invertase, thereby enabling the strain to utilize exogenous sucrose) is indicated by lowercase, boxed text. The initiator ATG and terminator TGA for ScSUC2 are indicated by uppercase italics, while the coding region is indicated with lowercase italics. The terminator region of the A. protothecoides enolase gene (ApPGH) gene is indicated by small capitals.

Construct pPB0014 was introduced into A. protothecoides PB5 using a modified lithium acetate transformation procedure. Briefly, 50 mL of seed growth medium containing 2% glucose in 250 mL Erlenmeyer flask was inoculated with a loop of cells from a plate. Cultures were grown for 2 days at room temperature on an orbital shaker at 140 rpm, to an OD 750 nm 2-4. 50 ml of cell culture was harvested in a Falcon tube and centrifuged for 5 minutes at 3750 rpm. The cell pellet was washed once with 5ml wash solution (0.1M Lithium acetate (LiAc) and 1× TE (10 mM Tris, 0.1 mM EDTA) buffer and centrifuged to discard the supernatant. The cells were resuspended in 500 μl wash solution, transferred to a sterile Eppendorf tube, and incubated for 1 hour on a rotary shaker at room temperature at 150 rpm. For each transformation, 150 μl of cell suspension was aliquoted into a 1.5 ml Eppendorf tube. 5-20 μg of linearized DNA was added to the cell suspension, then incubated for 30 minutes at room temperature at 150 rpm. 750 μl of PEG solution (0.1M LiAc, 1× TE, 40% PEG-4000) was added to the Eppendorf tube, and transformations were incubated overnight on the shaker at room temperature at 150 rpm. Cells were harvested by centrifugation at 5000 rpm for 30 seconds and resuspended in 250 μl of 1M sorbitol. About 180 μl of the transformation was spread on growth media plates with 1.5% agar with selection, using glass beads, and incubated at room temperature for 1 to 2 weeks. Single colonies were observed on the agar plate after 5-6 days of plating.

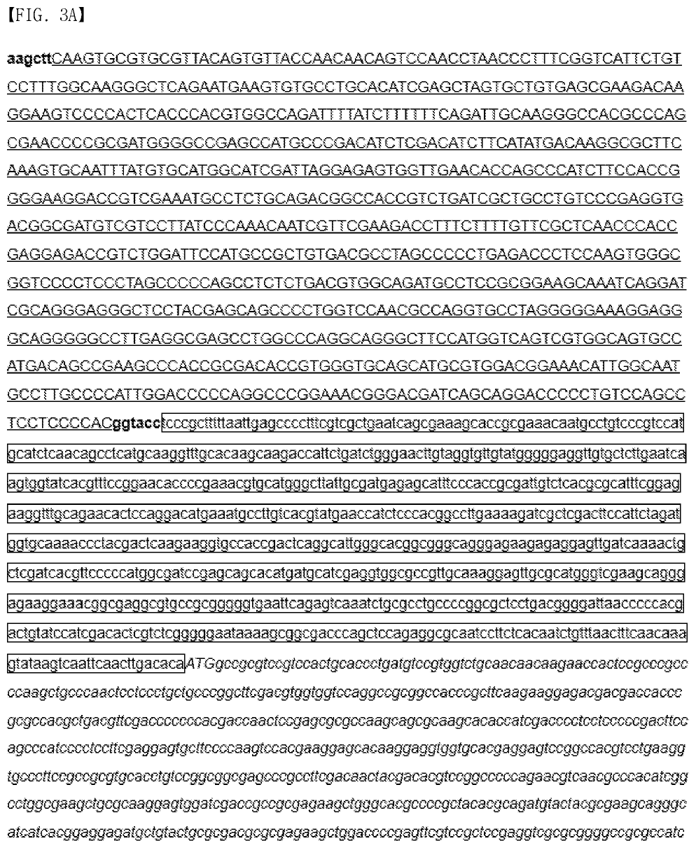

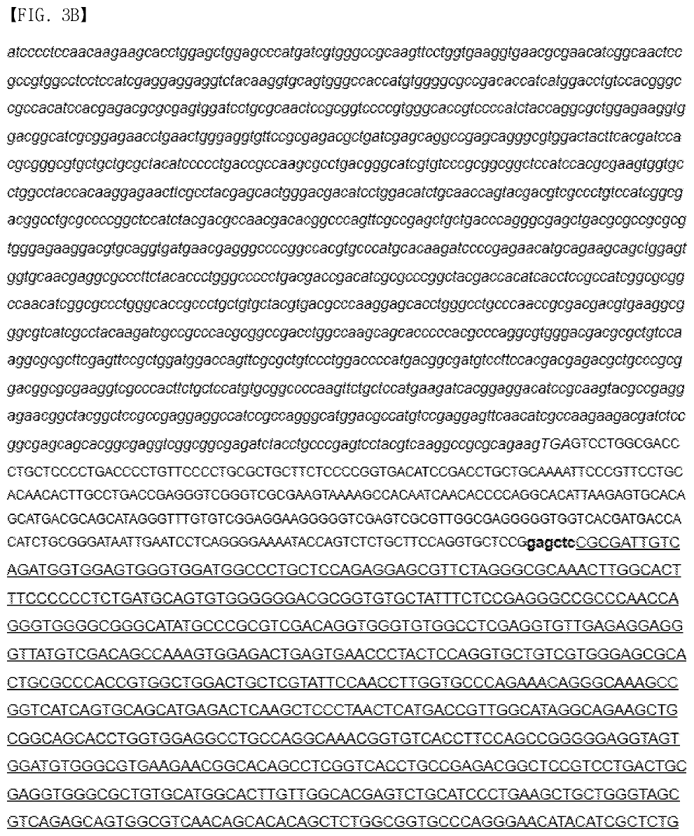

Primary transformants were selected for heterotrophic growth on media with sucrose as the sole carbon source. Colonies were clonally purified, and integration of pPB0014 at the LCYE-1 locus was verified for 11 strains by PCR amplification of the regions flanking the 5′ and 3′ ends of the integration site. Strain 14-2 was selected as the parent strain for subsequent modifications. Next, we disrupted LCYE-2, encoding the second allele of lycopene cyclase epsilon, with the goal of generating double knockout strains completely lacking in alpha-carotene-derived carotenoids. The sequence of the transforming DNA from the LCYE-2 disruption construct pPB0038 is shown below in FIG. 3. Relevant restriction sites used to generate linear DNA and for cloning are indicated in lowercase, bold, and are from 5′-3′ HindIII, KpnI, SacI, and HindIII. Underlined sequences at the 5′ and 3′ flanks of the construct represent genomic DNA from A. protothecoides that enable targeted integration of the transforming DNA via homologous recombination at the LCYE-2 locus. Proceeding in the 5′ to 3′ direction, the A. protothecoides HUP1 promoter (ApHUP1) driving the expression of the Arabidopsis thaliana THIC gene (AtTHIC, codon-optimized for expression in A. protothecoides and encoding 4-amino-5-hydroxymethyl-2-methylpyrimidine synthase activity, thereby permitting the strain to grow in the absence of exogenous thiamine) is indicated by lowercase, boxed text. The initiator ATG and terminator TGA for AtTHIC are indicated by uppercase italics, while the coding region is indicated with lowercase italics. The terminator region of the A. protothecoides heat shock protein 90 (ApHSP90) gene is indicated by small capitals.

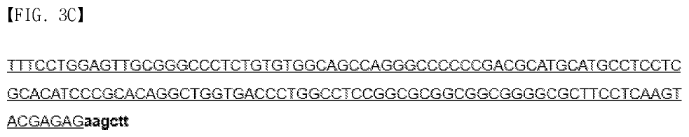

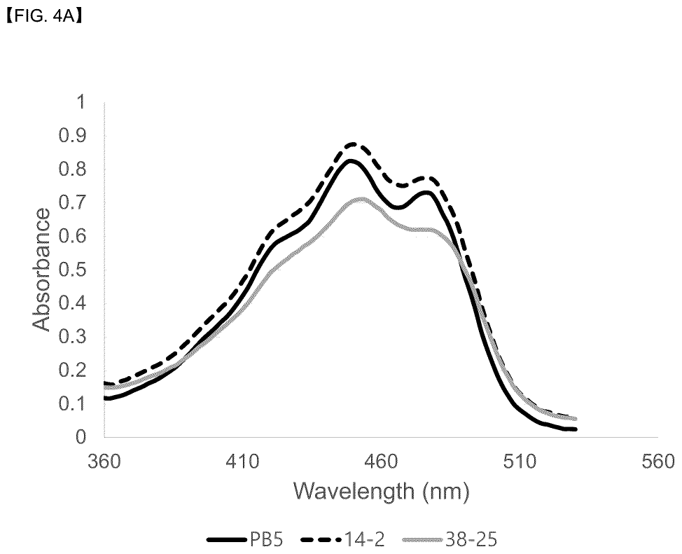

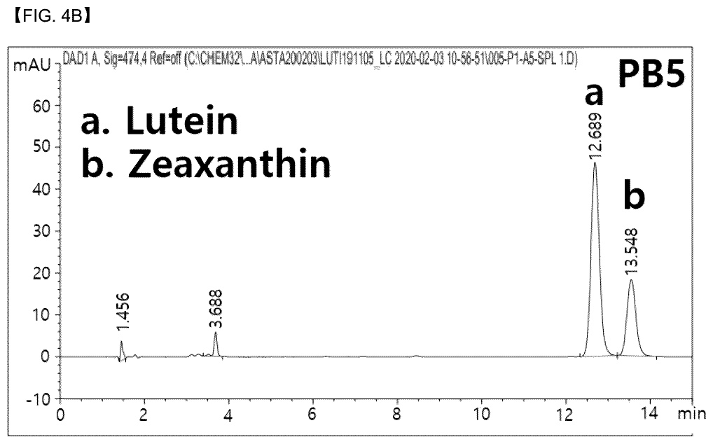

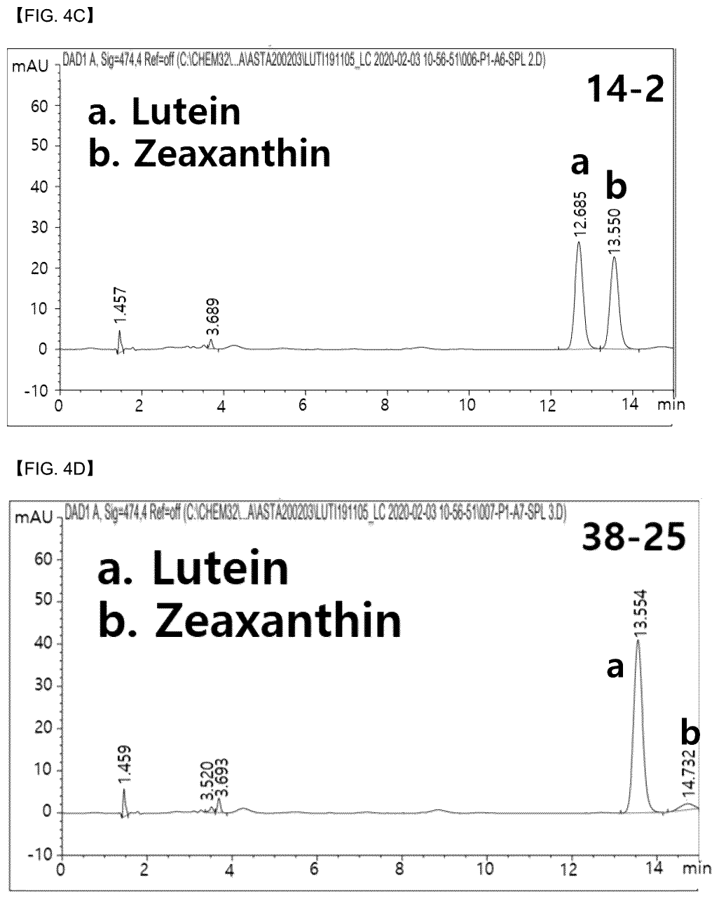

Construct pPB0038 was transformed into LCYE-1 disruption strain 14-2. Primary transformants were selected for heterotrophic growth on media without thiamine, and with sucrose as the sole carbon source. Colonies were clonally purified, and double knockout of both LCYE-1 and LCYE-2 was verified in strain 38-25 by PCR amplification of the regions flanking the 5′ and 3′ ends of the pPB0014 and pPB0038 integrations. Spectrophotometric, and HPLC analysis of carotenoid pigments from wild-type A. protothecoides PB5, 14-2, and 38-25 is shown in FIG. 4A and B. Pigments for spectrophotometric analysis were extracted by mechanical disruption of cell pellets mixed with 0.5 mm glass beads (BioSpec Products, Inc.) suspended in 80:20 acetone:methanol, using a Mini-Beadbeater-16 (BioSpec Products, Inc.). Absorption spectra were acquired using a Thermo Scientific GeneSys 10uv spectrophotometer. Lutein is the predominant pigment extracted from the wild-type strain. Reduction of lycopene cyclase epsilon activity by disrupting the LCYE-1 allele in 14-2 increases the ratio of zeaxanthin to lutein. Lutein accumulation is abolished in the 38-25 double knockout strain, and zeaxanthin is the major carotenoid.

Cell cultivation—Cells were grown in a growth medium composed of the following chemicals in 1 L deionized water. 0.025 g NaCl, 0.25 g NaNO₃, 0.074 g MgSO₄·7H₂O, 0.025 g CaCl₂·2H₂O, 0.075 g K₂HPO₄, 0.176 g KH₂PO₄, 2.38 g HEPES, 3 g Yeast extract, 5 ml stock vitamin solutions, and 20 g glucose. pH was adjusted to 6.8 with 20% NaOH. Stock vitamin solution contained the following in 200 ml HEPES solution (50 mM HEPES, pH7.8); 0.005 g Biotin, 0.44 g Thiamine HCl, 0.027 g B12, and 0.619 g D-Pantothenic acid hemicalcium. The stock vitamin solution was filtered, sterilized, and stored at 4° C. refrigerator.

Seed flask cultivation—in 250 ml Erlenmeyer flask containing 95 ml growth medium and 5 ml 40% stock glucose solution, loopful of cells were inoculated from the agar plate and grown for 2 days on an orbital shaker at 115 rpm at 28° C. under 0.1-1 μmol photons/m² s low intensity LED light.

Main flask cultivation—in 1 L Erlenmeyer flask containing 332.5 ml growth medium and 17.5 ml 40% stock glucose solution, inoculate cells from seed flask so that initial optical density (OD) at 750 nm wavelength was 0.5-0.6. Incubate the flask on the orbital shaker under 0.1-1 μmol photons/m² s low intensity LED light at 115 rpm at 28° C. for 4-5 days until all glucose is exhausted. Harvest the cells via centrifugation and freeze dry the cell pellet. Store the freeze-dried cell pellet at −20° C. freezer.

The lipid extraction method and HPLC parameters are described below.

About 3-5 g lyophilized PB5 and modified strain cell powders were finely ground in a mortar using a pestle. 30 ml of ether was added to the milled microalga powders then vortexed for about 30 seconds to extract oil and carotenoids. 30 ml of additional ether was used if necessary to complete the extraction of all the oil and carotenoids. The remaining ether was evaporated using an evaporator at room temperature. The extracted oil was dissolved and diluted at a 1:10 ratio with a mixed Hexane:Ethyl Acetate (70:30, v/v) solvent. 10 μl of the diluted solution was injected into an HPLC (Agilent 1260 Infinity II, Agilent, USA) and separation of analytes was conducted on a Luna silica 100A column (25 cm×4.6 mm; 5 μm, Phenomenex, USA). The mobile phase consisted of hexane: ethyl acetate (70:30, v/v) at a flow rate of 1.5 ml/min. The column temperature was set at 30° C. and lutein, zeaxanthin, and standards were detected at 446 nm. The lutein standard eluted at a retention time of 12.6 minutes and the zeaxanthin standard eluted at a retention time of 13.5 minutes. Table 1 summarized the results of carotenoid analysis of each genetically modified strain and wild-type strain PB5 (control).

TABLE 1
Carotenoids identified in extracted oil
from PB5 and modified strains via HPLC.

| Strains | Carotenoids | Retention time | Percent (%) |
|---|---|---|---|
| PB5 (Control) | Lutein | 12.689 | 67.0392 |
| | Zeaxanthin | 13.548 | 27.9272 |
| 14-2 | Lutein | 12.685 | 50.1228 |
| | Zeaxanthin | 13.55 | 45.4815 |
| 38-25 | Zeaxanthin | 13.554 | 87.8917 |

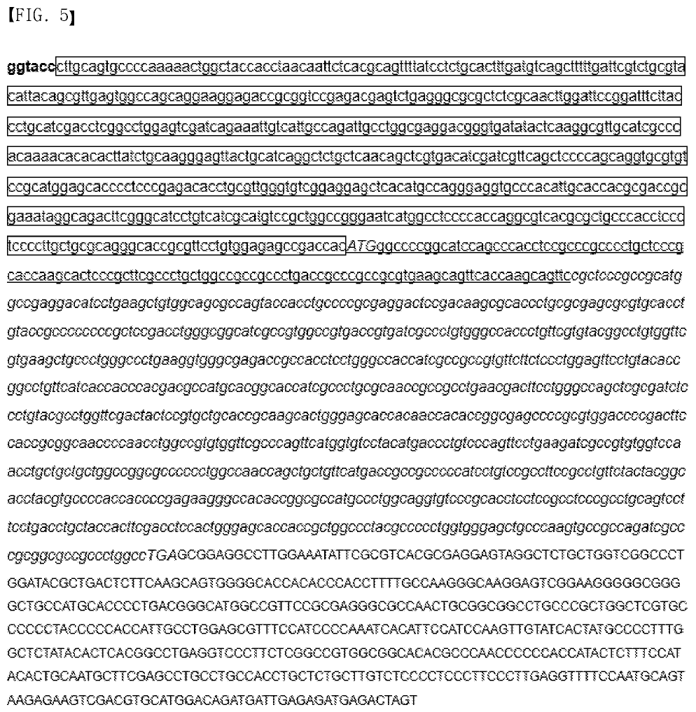

Next, we sought to express beta-carotene ketolase activity in A. protothecoides cells, allowing them to make keto-carotenoids with high value as antioxidants. To achieve this, we introduced an expression module for the C. reinhardtii beta-carotene ketolase gene 1 (CrBKT1) into the pPB0038 backbone. The sequence of the expression module cloned into the KpnI site of pPB0038 to generate construct pPB0120 is shown below in FIG. 5. KpnI and SpeI sites flanking the expression module and used for cloning are indicated in lowercase, bold. Proceeding in the 5′ to 3′ direction, the A. protothecoides SAD2 promoter (ApSAD2) driving the expression of the CrBKT1 gene is indicated by lowercase, boxed text. The initiator ATG and terminator TGA for CrBKT1 are indicated by uppercase italics, while the coding region is indicated with lowercase italics. The sequence encoding the predicted plastid transit peptide is underlined. The terminator region of A. protothecoides SAD2 is indicated by small capitals.

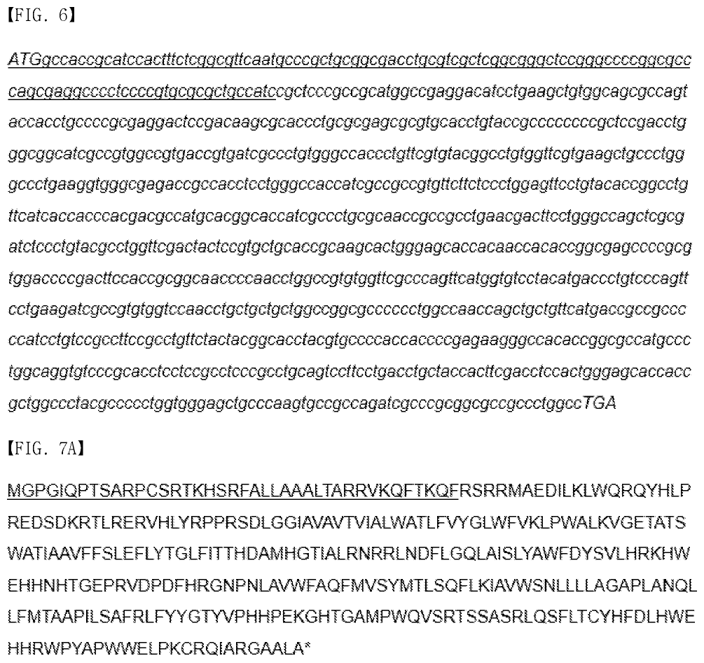

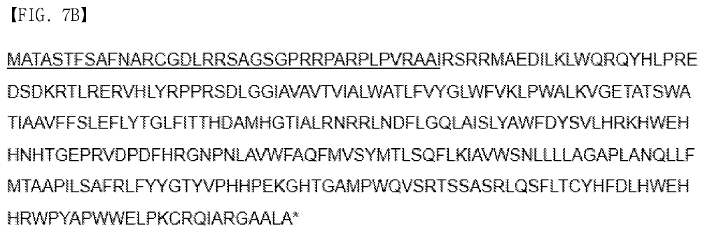

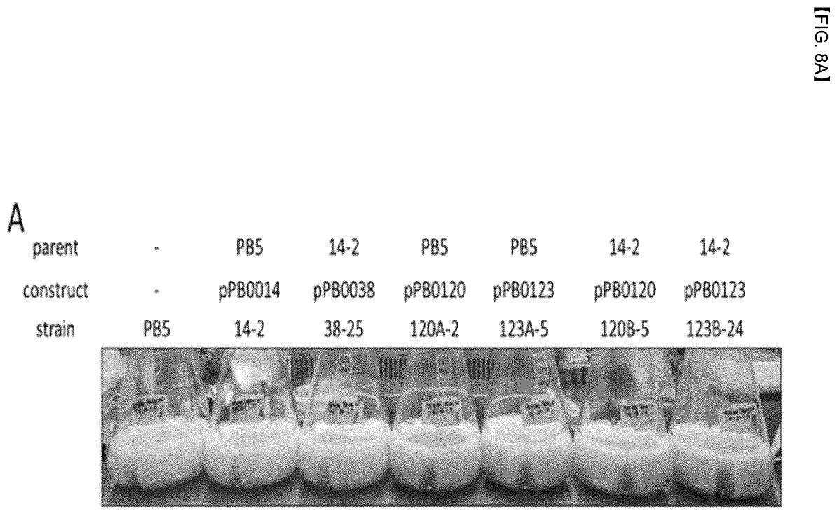

We considered that the heterologous CrBKT1 protein might not be imported efficiently into Auxenochlorella plastids, so we also made a version of the CrBKT1 expression module that replaced the sequence encoding the native plastid transit peptide with the corresponding sequence from the endogenous Auxenochlorella SAD2 gene. The sequence of the chimeric ApSAD2tp_CrBKT1 coding sequence in construct pPB0123 is shown below in FIG. 6. pPB0123 was otherwise identical to pPB0120 and targeted the AtTHIC transformation marker and the CrBKT1 expression module to the LCYE-2 locus. The sequences of the native CrBKT1 protein and the ApSAD2tp_CrBKT1 chimeric proteins are shown in FIGS. 7A and 7B, respectively.

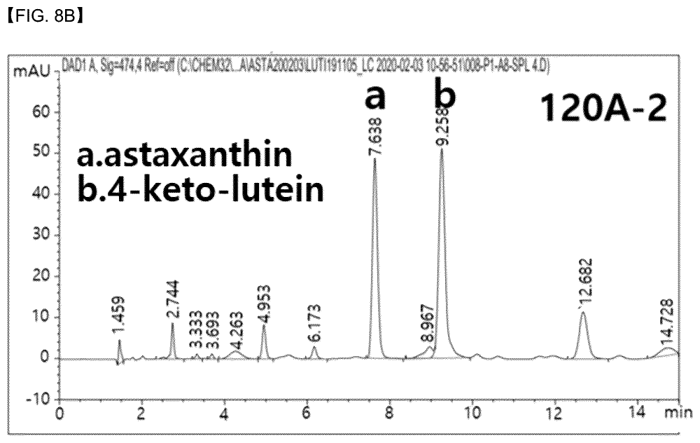

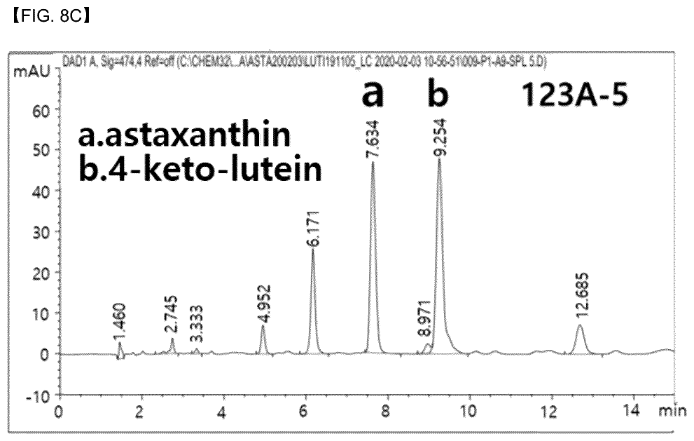

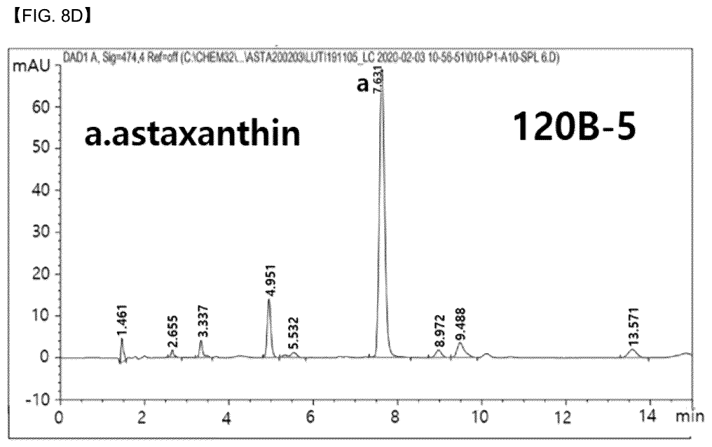

Constructs pPB0120 and pPB0123 were both transformed into wild-type A. protothecoidesPB5 to make LCYE-2 single allele knockouts expressing CrBKT1, and into LCYE-1 disruption strain 14-2 to make LCYE double knockouts expressing CrBKT1. Primary transformants made in the wild-type A. protothecoides background were selected for heterotrophic growth on glucose-containing media without thiamine, while transformants generated in the 14-2 parent strain were selected on media without thiamine, supplemented with sucrose. Primary transformants were clonally purified and shake flask cultures of representative strains are shown in FIG. 8A. Strains transformed with pPB0123, expressing chimeric ApSAD2tp_CrBKT1 produced more red-colored ketocarotenoids than pPB0120 transformants that expressed native CrBKT1, suggesting that the chimeric protein was imported and processed more efficiently than the native protein. HPLC analysis, shown in FIG. 8B, showed that the predominant ketocarotenoids in LCYE-2 single allele knockouts (A strains, wild-type A. protothecoides parent) were 4-keto-lutein and astaxanthin, derived from lutein and zeaxanthin, respectively. The strains that expressed CrBKT1 with both LCYE alleles disrupted (B strains) made astaxanthin almost exclusively.

The lipid extraction method and HPLC parameters for keto-carotenoids analysis-Ether extraction of oil and carotenoids was performed as described above. The extracted oil was dissolved and diluted at a 1:100 ratio with a solvent mixture of Hexane:Acetone (82:18, v/v). 20 μl of the diluted solution was injected into an HPLC (Agilent 1260 Infinity II, Agilent, USA) and separation of analytes was conducted on a Luna silica column (15 cm×4.6 mm; 3 μm, Phenomenex, USA). The mobile phase consisted of Hexane:Acetone (82:18, v/v) ran at a flow rate of 1.2 ml/min. The column temperature was not set and astaxanthin and standards were detected at 474 nm. The astaxanthin standard was eluted at a retention time of 7.6 minutes. Table 2 summarized HPLC analysis results of genetically modified strains producing different carotenoids.

TABLE 2
Carotenoids identified in extracted
oil from modified strains via HPLC.

| Strains | Carotenoids | Retention time | Percent (%) |
|---|---|---|---|
| 120A-2 | Astaxanthin | 7.638 | 29.8938 |
| | Keto Lutein | 9.258 | 39.4228 |
| | Lutein | 12.682 | 11.5903 |
| 123A-5 | Astaxanthin | 7.634 | 30.0058 |
| | Keto Lutein | 9.254 | 39.9874 |
| | Lutein | 12.685 | 7.5039 |
| 120B-2 | Astaxanthin | 7.631 | 71.5094 |
| | Zeaxanthin | 13.571 | 3.3719 |
| 123B-24 | Astaxanthin | 7.632 | 77.876 |
| | Zeaxanthin | 13.582 | 2.5516 |

### EXAMPLE 2. PRODUCTION OF STRAINS WITH ACCUMULATING SQUALENE

Squalene is used extensively in cosmetics as an emollient and moisturizer, while its antioxidant properties are exploited in sunscreens and anti-aging products. There are numerous pharmacological applications, including use in formulations as a chemoprotective agent, an anti-bacterial and anti-fungal agent, an adjuvant for vaccines, and a drug carrier. In vivo studies have demonstrated its value as a food supplement with benefits for weight and cholesterol control. Squalene is also a valuable precursor for the chemical synthesis of steroids and other bioactive molecules.

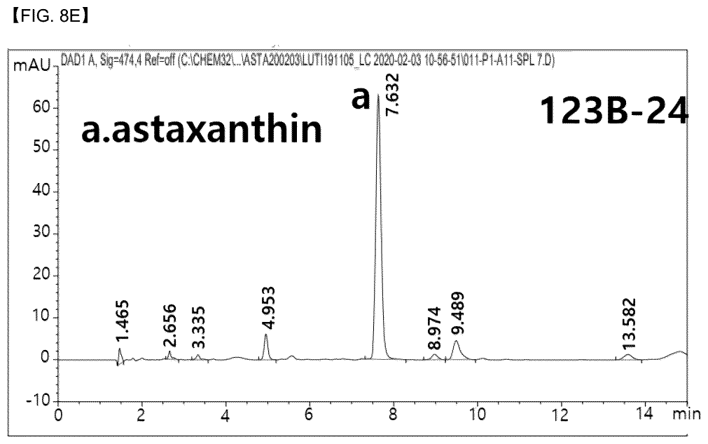

We have enhanced the value of A. protothecoides, PB5, storage lipid by engineering strains to accumulate squalene during the lipid production phase by targeted disruption of the squalene epoxidase (SQE) gene. Squalene epoxidase catalyzes the squalene cyclization step in phytosterol biosynthesis. Knockout of the equivalent ERG1 gene in S. cerevisiae led to squalene accumulation. Similar metabolic engineering strategies have been applied in cyanobacteria and purple non-sulfur bacteria, and elevated squalene levels were observed in C. reinhardtii strains where RNAi was used to knock-down SQE gene expression. In this example, we describe genetically engineered A. protothecoides, PB5 strains in which we have disrupted both alleles of SQE. These modifications block downstream phytosterol biosynthesis and cause the accumulation of squalene in cellular lipids. The sequence of the transforming construct pPB0065, targeting disruption of ApSQE-2 is provided in FIG. 9. Restriction sites are indicated with bold, lowercase text. HindIII sites delimit the 5′ and 3′ ends of the transforming DNA. EcoRI and SacI sites flank the selection cassette. Underlined sequences represent SQE-2 genomic DNA targeting integration at the SQE-2 locus via homologous recombination. Proceeding from 5′ to 3′, the selection cassette contains the ApHUP1 promoter (lowercase, boxed text), driving the expression of codon-optimized AtTHIC. The initiator ATG and terminator TGA for AtTHIC are indicated by uppercase italics, while the coding region is indicated with lowercase italics. The ApHSP90 terminator region is indicated by small capitals.

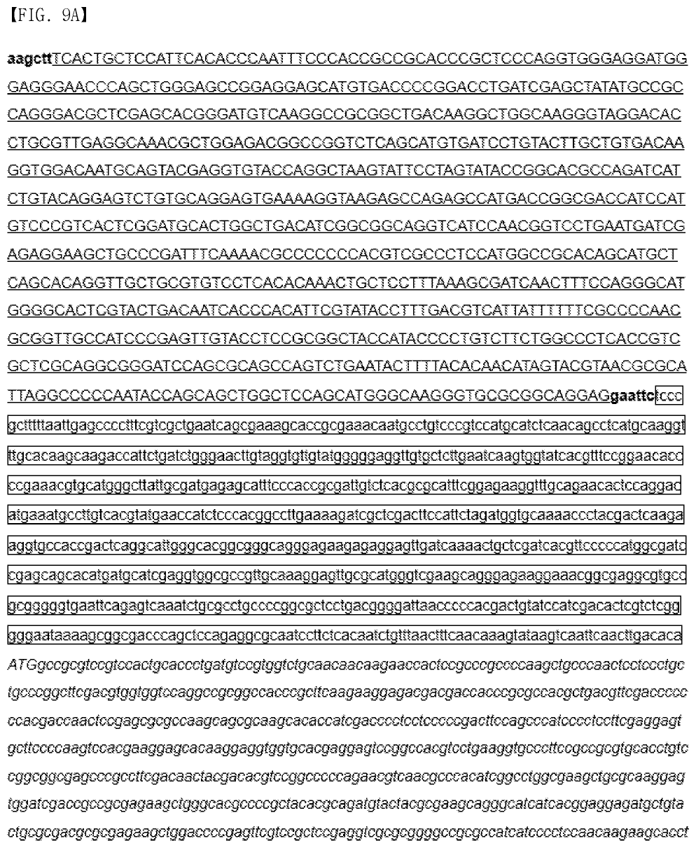

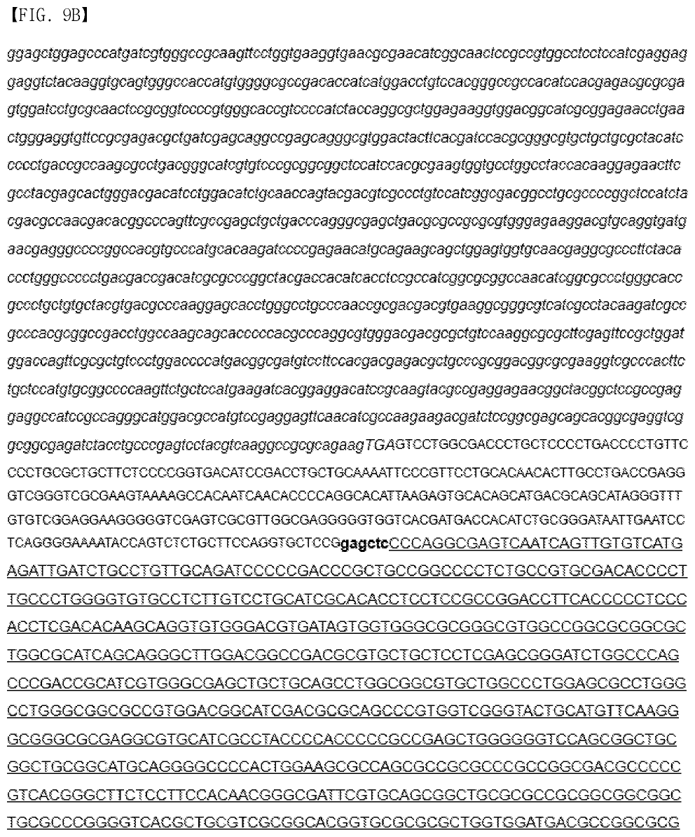

SQE-2 knockout strains were generated by transformation of pPB0065 into A. protothecoides PB5. Primary transformants were selected on glucose-containing growth media without thiamine. Colonies were clonally purified, and targeted disruption of SQE-2 was verified by PCR amplification and sequencing of the regions flanking the integration site. Transformant 65-4 was selected as the parent strain for subsequent transformation with construct pPB0077, targeting ApSQE-1. The sequence of the transforming DNA from pPB0077 is shown below in FIG. 10. Restriction sites are indicated with bold, lowercase text. HindIII sites delimit the 5′ and 3′ ends of the transforming DNA. EcoRI and SacI sites flank the selection cassette. Underlined sequences represent ApSQE-1 genomic DNA targeting integration at the ApSQE-1 allele via homologous recombination. Proceeding from 5′ to 3′, the selection cassette contains the CrTUB2 promoter (lowercase, boxed text), driving the expression of codon-optimized ScSUC2. The initiator ATG and terminator TGA for ScSUC2 are indicated by uppercase italics, while the coding region is indicated with lowercase italics. The ApPGH terminator region is indicated by small capitals.

Primary transformants from the introduction of pPB0077 into strain 65-4 were selected on growth media with sucrose and without thiamine. Colonies were clonally purified and targeted disruption of both ApSQE-1 and ApSQE-2 was verified by PCR amplification and sequencing of the regions flanking the integration site.

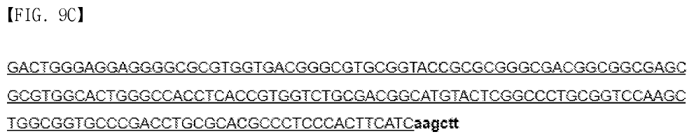

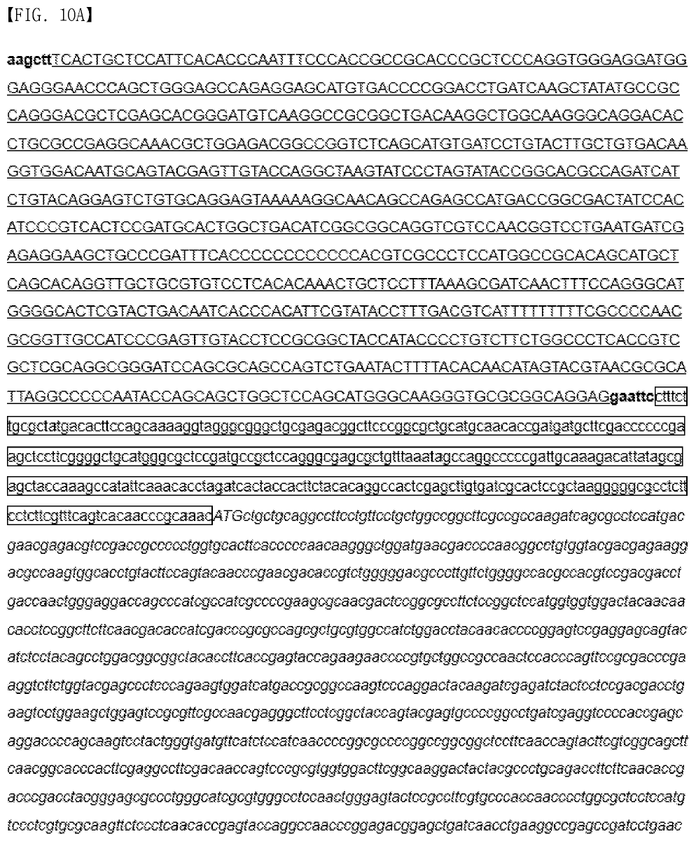

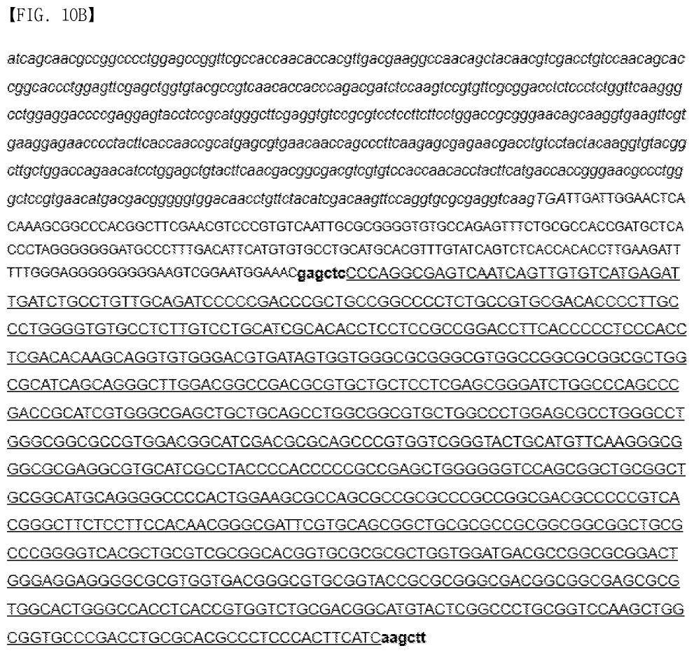

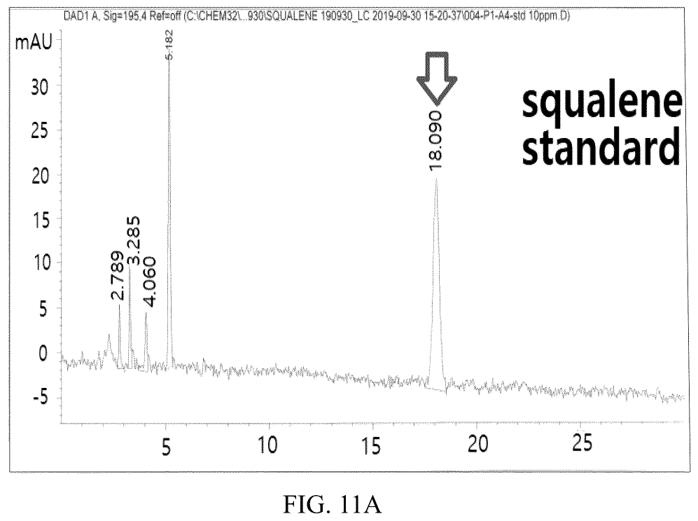

A representative strain, 77B-21, was grown in lipid production media and the squalene content of crude lipid extracts was measured by HPLC (FIG. 11).

Cell cultivation conditions—Cells were grown in a growth medium containing the followings in 1 L deionized water: 4.2 g K₂HPO₄, 3.57 g NaH₂PO₄·H₂O, 0.24 g MgSO₄·7H₂O, 0.025 g CaCl₂·2H₂O, 0.25 g citric acid, 2 micromolar thiamine-HCl, and 10 mL of trace metal solution. 1 L of trace metal solution contained 2.75 g citric acid, 0.011 g CuSO₄·5H₂O, 0.081 g CoCl₂·6H₂O, 0.33 g H₃BO₃, 1.4 g ZnSO₄·7H₂O, 0.9485 g MnCl₂·4H₂O, 0.039 g Na₂MoO₄·2H₂O, 0.11 g FeSO₄·7H₂O, 0.0144 g NiSO₄·6H₂O. Pre-seed and seed medium contained 0.991 g/L of (NH₄)₂SO₄, and lipid production media contained 0.248 g of (NH₄)₂SO₄. The pre-seed medium was supplemented with 5 g/L glucose; seed medium contained 20 g/L glucose and lipid production contained 40 g/L glucose.

The lipid extraction method and HPLC parameters for squalene analysis—About 3 g of lyophilized PB5 and modified strain cell powders were finely ground in a mortar using a pestle. 30 ml of ether was added to the ground PB5 powder and the resulting suspension was vortexed for about 30 seconds to extract oil. An additional 30 ml of ether was used to complete oil extraction. The remaining ether was evaporated at room temperature using an evaporator. The extracted oil was dissolved and diluted at a 1:10 ratio with a solvent mixture solvent of Acetonitrile:Methanol (8:2, v/v), which was also used as the mobile phase. 30 μl of the diluted solution was injected into an HPLC (Agilent 1260 Infinity II) and separation of analytes was conducted on a C18 reverse column (15 cm×4.6 mm; Shiseido, Japan). The mobile phase's flow rate was 1.5ml/min. The column temperature was set at 35° C. and squalene and the standard was detected at 195nm. Squalene standard eluted at a retention time of 18.090.

Squalene accumulated to 1222 ppm in oil from strain 77B-21, a level that is on par with the squalene content of olive oil, a common commercial source.

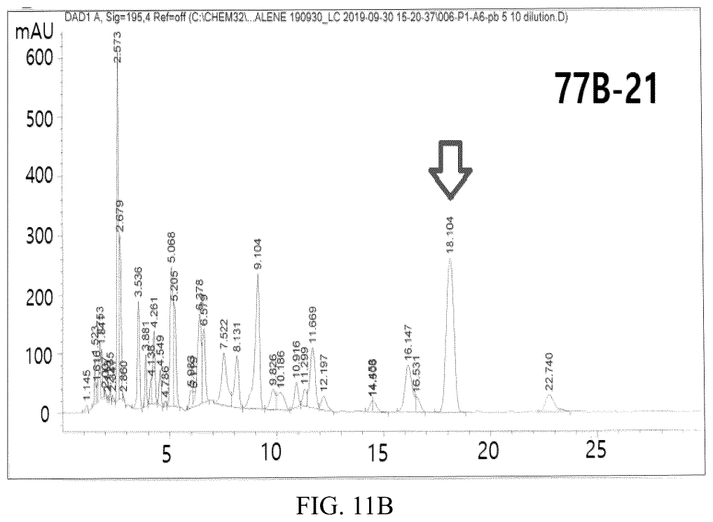

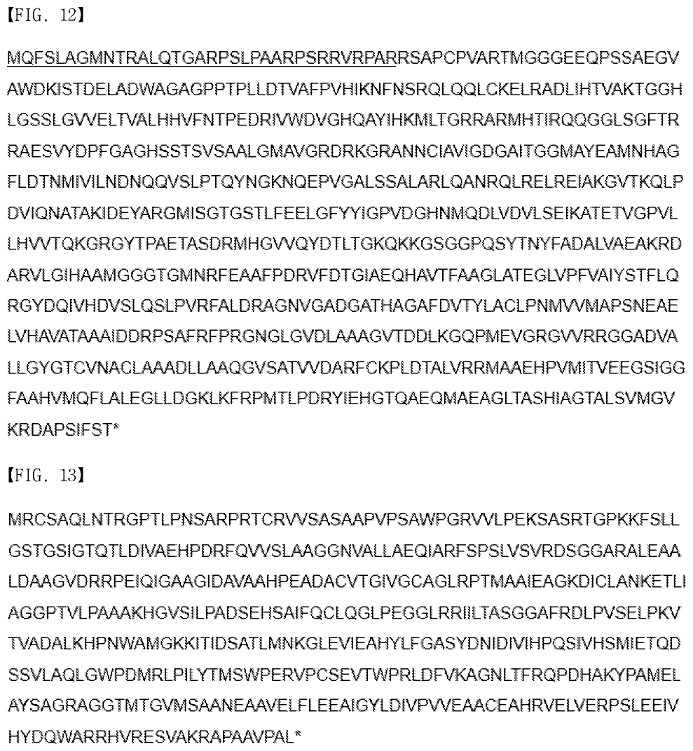

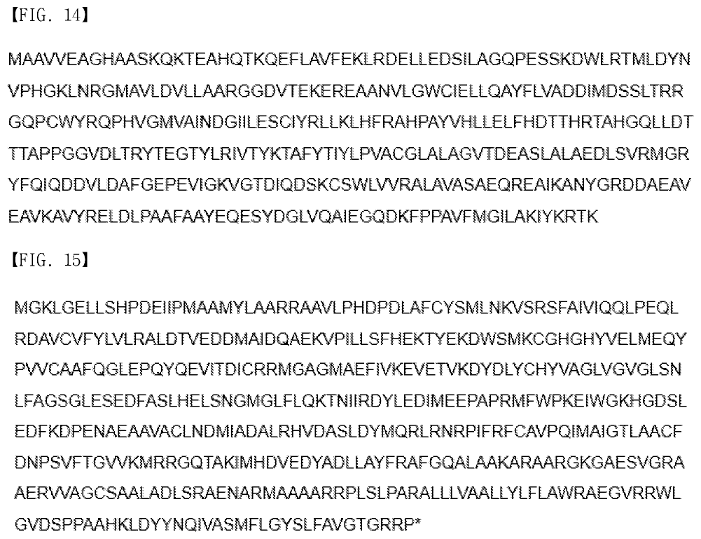

The value of A. protothecoides oil would be enhanced by further increases in squalene levels. This may be achieved through overexpression of key enzymes in the isoprenoid biosynthesis pathway to increase flux. The amino acid sequences of A. protothecoides 1-deoxy-D-xylulose 5-phosphate synthase (DXS), 1-deoxy-D-xylulose 5-phosphate reductoisomerase (DXR), farnesyl diphosphate synthase (FDPS), and squalene synthase are provided in FIGS. 12-15. Overexpression of the native A. protothecoides enzymes, or of heterologous enzymes from plants or other algae, in the SQE double knockout background may improve squalene accumulation.

### EXAMPLE 3. PRODUCTION OF STRAINS WITH INCREASED LEVELS OF OMEGA-3 AND OMEGA-6 FATTY ACIDS

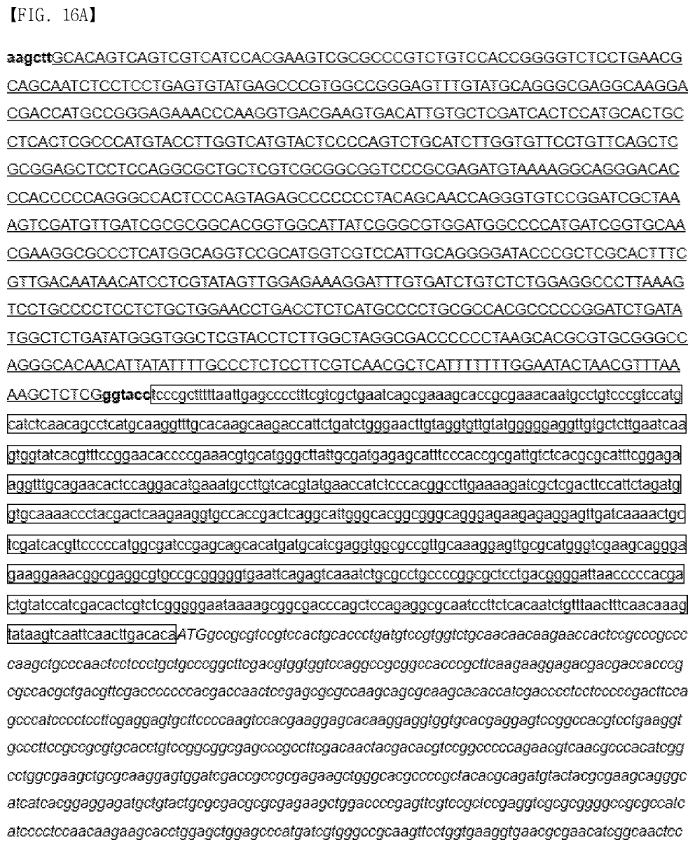

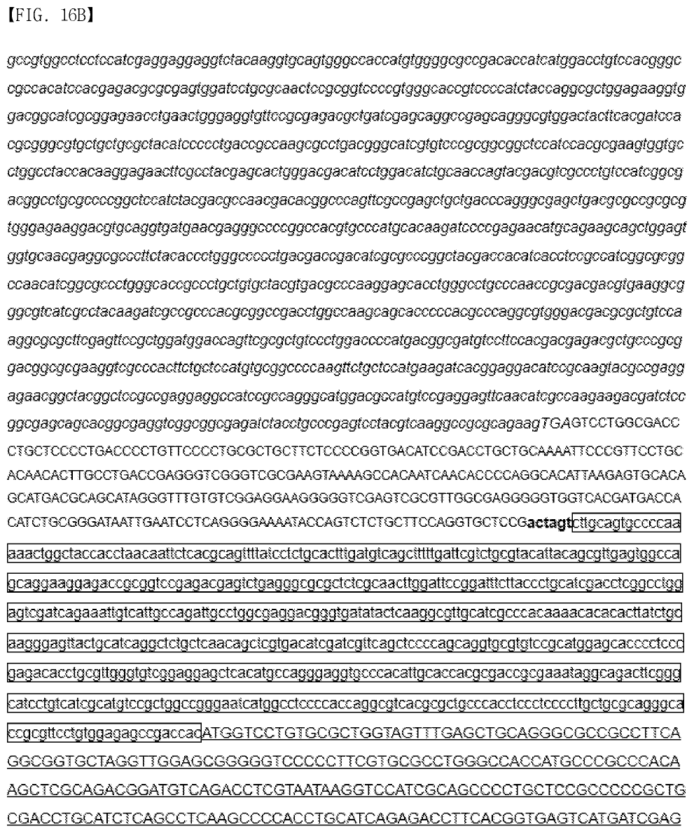

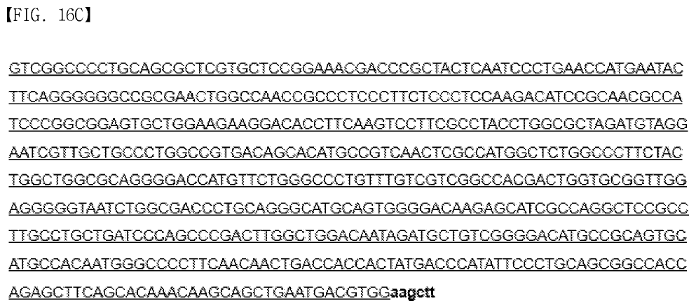

The fatty acid composition of A. protothecoides storage lipid is typical for Trebouxiophyte algae, consisting mainly of oleic (˜68%), palmitic (˜12%), and linoleic (˜13%) acids, with minor amounts of stearic, myristic, α-linolenic, and palmitoleic acids. We sought to improve the nutritional quality of A. protothecoides triacylglyceride oils by increasing the polyunsaturated fatty acid (PUFA) content. First, we activated the endogenous FAD3 gene, encoding fatty acid desaturase 3, by swapping the native FAD3 promoter with promoters from genes that are strongly upregulated during lipid production. FAD3 introduces a double bond at the Δ15 position of linoleic acid (C18:2) to make α-linolenic acid. The sequence of the transforming construct pPB0039, targeting insertion of the ApSAD2 promoter upstream of the FAD3-1 coding sequence is provided in FIG. 16. Restriction sites are indicated with bold, lowercase text. HindIII sites delimit the 5′ and 3′ ends of the transforming DNA. KpnI and SpeI sites flank the selection cassette. Underlined sequences represent FAD3-1 genomic DNA targeting integration at the FAD3-1 locus via homologous recombination. Proceeding from 5′ to 3′, the selection cassette contains the ApHUP1 promoter (lowercase, boxed text), driving the expression of codon-optimized AtTHIC. The initiator ATG and terminator TGA for AtTHIC are indicated by uppercase italics, while the coding region is indicated with lowercase italics. The ApHSP90 terminator region is indicated by small capitals. Lowercase, boxed text delineates the ApSAD2 promoter.

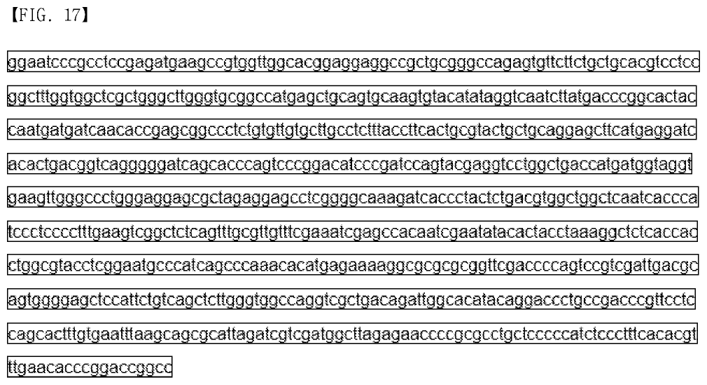

We also made a version of the FAD3 integration construct expression module that replaced the native FAD3 promoter with the promoter from the A. protothecoides FATA gene, encoding the acyl-ACP thioesterase. Construct pPB0041 was identical to pPB0039 except that ApFATA promoter shown below in FIG. 17 in lowercase, boxed text, was used to drive the expression of FAD3.

pPB0039 and pPB0041 were transformed into A. protothecoides PB5 and primary transformants were selected on glucose-containing growth media without thiamine. Colonies were clonally purified and insertion of the constructs at the FAD3 locus was verified by PCR amplification and sequencing of the regions flanking the integration site.

Cell cultivation conditions-Cells were grown in a growth medium composed of the following chemicals in 1 L of deionized water: 4.2 g K₂HPO₄, 3.57 g NaH₂PO₄·H₂O, 0.24 g MgSO₄·7H₂O, 0.025 g CaCl₂·2H₂O, 0.25 g citric acid, 2 micromolar thiamine-HCl, and 10 mL of trace metal solution. 1 L of trace metal solution contained 2.75 g citric acid, 0.011 g CuSO₄·5H₂O, 0.081 g CoCl₂·6H₂O, 0.33 g H₃BO₃, 1.4 g ZnSO₄·7H₂O, 0.9485 g MnCl₂·4H₂O, 0.039 g Na₂MoO₄·2H₂O, 0.11 g FeSO₄·7H₂O, 0.0144 g NiSO₄·6H₂O. Pre-seed and seed medium contained 0.991 g/L of (NH₄)₂SO₄, and lipid production media contained 0.248 g of (NH₄)₂SO₄. The pre-seed medium was supplemented with 5 g/L glucose; seed medium contained 20 g/L glucose and lipid production contained 40 g/L glucose.

Freeze-dried microalgal samples were sent to Microbial ID Inc., (Delaware, USA) for fatty acid methyl ester analysis (FAME). Microbial ID Inc. utilized standardized gas chromatographic analysis of fatty acid methyl esters method developed by MIDI Inc., (Delaware, USA) and the reference could be found in their technical note #101, published and revised in 2006. The fatty acid profiles of lipids from shake flask assays of representative strains are shown in Table 3. Strains driving expression of one allele of FAD3 with the ApSAD2 promoter (39-1 and 39-9 strains) or the ApFATA promoter (41-1 and 41-3 strains) showed a slight increase in α-linolenic acid (ALA, omega-3) from 2% in the wild-type (PB5) to about 3%. The increase in ALA came at the expense of linoleic acid (omega-6), improving the omega-6: omega-3 ratio from 7 to about 4 (lower is better).

TABLE 3
Fatty acid profiles as a percentage of total fatty acids for
A. protothecoides strains transformed with pPB0039 or pPB0041.

| strain | PB5 | 39-1 | 39-9 | 41-1 | 41-3 |
|---|---|---|---|---|---|
| C14:0 myristic | 1.7 | 1.6 | 1.7 | 1.6 | 1.7 |
| C16:0 palmitic | 12.7 | 13.0 | 12.6 | 12.0 | 12.6 |
| C16:1n-7 palmitoleic | 0.3 | 0.3 | 0.3 | 0.3 | 0.3 |
| C18:0 stearic | 2.5 | 2.5 | 2.6 | 2.4 | 2.3 |
| C18:1n-9 oleic | 66.3 | 68.9 | 67.8 | 68.5 | 67.5 |
| C18:2n-6 linoleic LA | 14.2 | 10.5 | 11.1 | 11.7 | 11.9 |
| C18:3n-3 α-linolenic | 2.0 | 2.9 | 3.3 | 3.0 | 3.1 |
| omega-6:omega-3 | 6.9 | 3.6 | 3.4 | 4.0 | 3.9 |
| % PUFA | 16.2 | 13.4 | 14.4 | 14.6 | 15.0 |

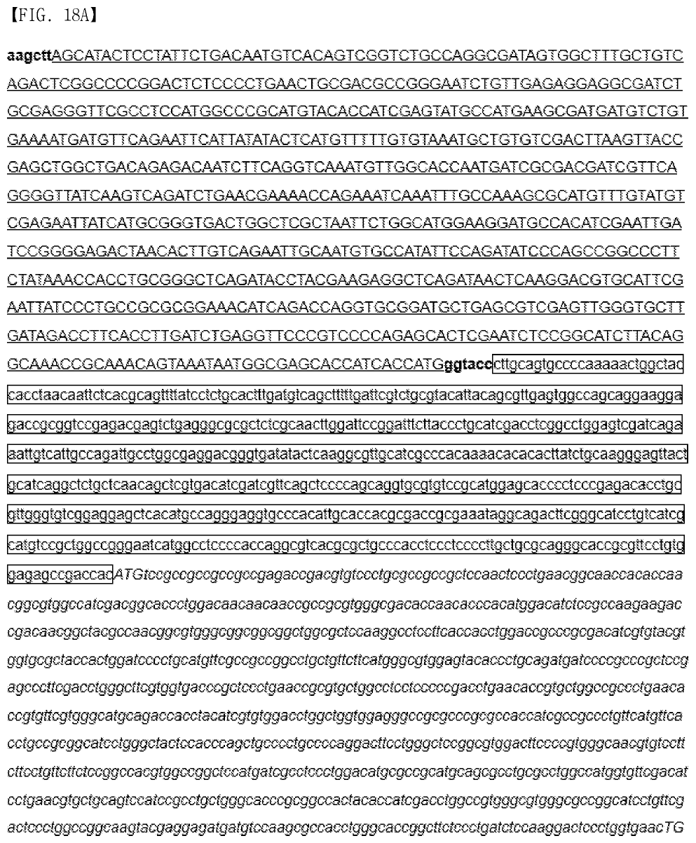

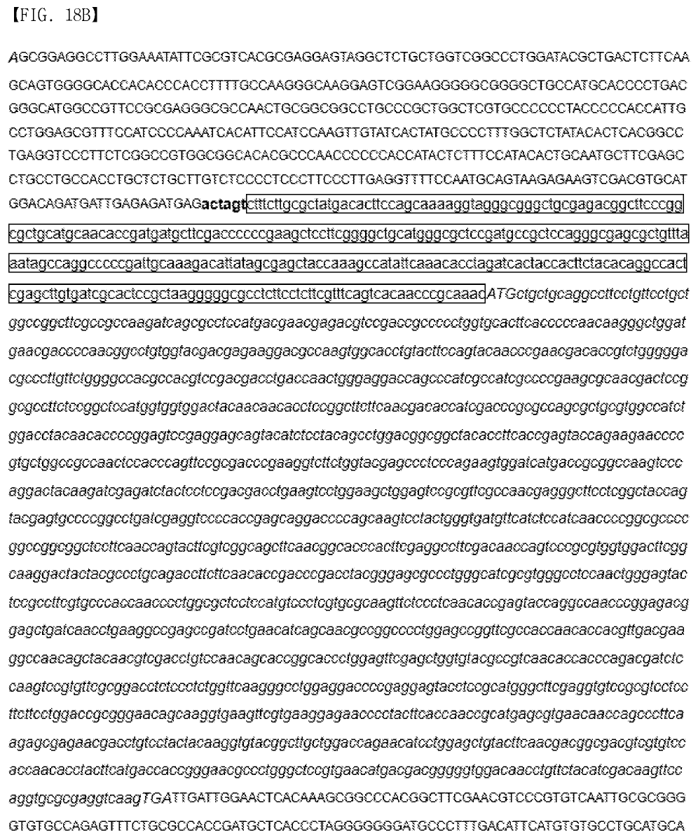

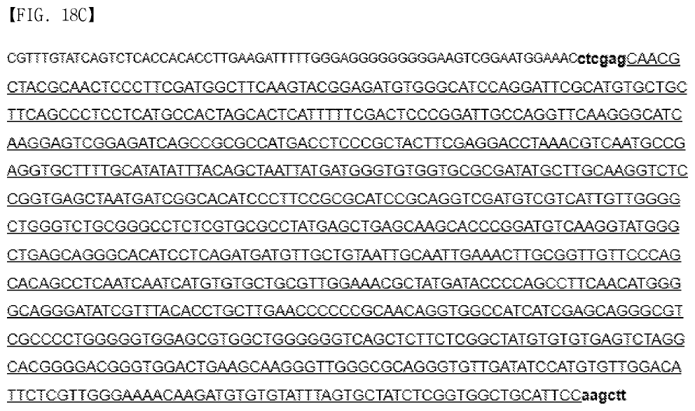

The Δ9 double bond in C18:1 is introduced by the stearoyl-ACP desaturases (SADs) in the plastid. Formation of the Δ12 and Δ15 double bonds, catalyzed by FAD2 and FAD3, respectively, occurs in the endoplasmic reticulum, and these enzymes use membrane lipids as their substrate. The relatively low abundance of C18:2 and C18:3 α in wild-type A. protothecoides storage lipid results from the competition between the acyltransferases of the Kennedy pathway for the formation of TAG, and the enzymes of the Lands cycle, which control the exchange of fatty acids between diacylglycerol (DAG) and membrane phospholipids. We considered that increasing the transfer of C18:1 between DAG and phospholipids might improve PUFA production, so we introduced a construct to overexpress Arabidopsis PDCT, encoding phosphatidylchloline:diacylglycerol choline phosphotransferase into strain 41-3. The sequence of the transforming construct pPB0118, targeting AtPDCT and ScSUC2 expression to the THI4 locus is provided in FIG. 18. Restriction sites are indicated with bold, lowercase text. HindIII sites delimit the 5′ and 3′ ends of the transforming DNA. KpnI, SpeI, and XhoI sites separate the AtPDCT expression module and the ScSUC2 expression cassette. Underlined sequences represent THI4 genomic DNA targeting integration at the THI4 locus via homologous recombination. Proceeding from 5′ to 3′, the selection cassette contains the ApSAD2 promoter (lowercase, boxed text), driving the expression of codon-optimized AtPDCT. The initiator ATG and terminator TGA for AtPDCT are indicated by uppercase italics, while the coding region is indicated with lowercase italics. The ApSAD2 terminator region is indicated by small capitals. Lowercase, boxed text delineates the CrTUB2 promoter, driving the expression of codon-optimized ScSUC2. The initiator ATG and terminator TGA for ScSUC2 are indicated by uppercase italics, while the coding region is indicated with lowercase italics. The ApPGH terminator region is indicated by small capitals.

pPB0118 was transformed into strain 41-3, and primary transformants were selected on sucrose-containing growth media without thiamine. Colonies were clonally purified and insertion of the constructs at the THI4 locus was verified by PCR amplification and sequencing of the regions flanking the integration site. Freeze-dried microalgal cell samples were sent to Microbial ID inc. (Delaware, USA) for fatty acid methyl ester analysis and the profiles of lipids from shake flask assays of representative strains containing the pPB0118 construct are shown in Table 4. C18:2 accumulation increased by 2.5-fold in strains 118B-8 and 118B-20, indicating that expression of AtPDCT during lipid production caused significant enhancement of FAD2 activity. ALA levels only increased by about 1% compared to the 41-3 parent strain, suggesting that incorporation of C18:2 into TAG was favored over desaturation by FAD3. The omega-6: omega-3 ratio increased from 3.8 in 41-3 to above 8, due to the increase in C18:2 without a concomitant increase in ALA.

TABLE 4
Fatty acid profiles as a percentage of total fatty
acids for strain 41-3 transformed with pPB0118.

| strain | PB5 | 41-3 | 118B-8 | 118B-20 |
|---|---|---|---|---|
| C14:0 myristic | 1.7 | 1.8 | 2.1 | 2.1 |
| C16:0 palmitic | 12.3 | 12.5 | 12.4 | 13.3 |
| C16:1n-7 palmitoleic | 0.3 | 0.3 | 0.3 | 0.4 |
| C18:0 stearic | 2.4 | 2.3 | 1.8 | 2.2 |
| C18:1n-9 oleic | 67.9 | 67.5 | 45.4 | 46.4 |
| C18:2n-6 linoleic LA | 13.1 | 12.0 | 33.3 | 30.8 |
| C18:3n-3 α-linolenic | 1.8 | 3.1 | 4.0 | 3.9 |
| omega-6:omega-3 | 7.2 | 3.8 | 8.3 | 8.0 |
| % PUFA | 15.0 | 15.1 | 37.3 | 34.7 |

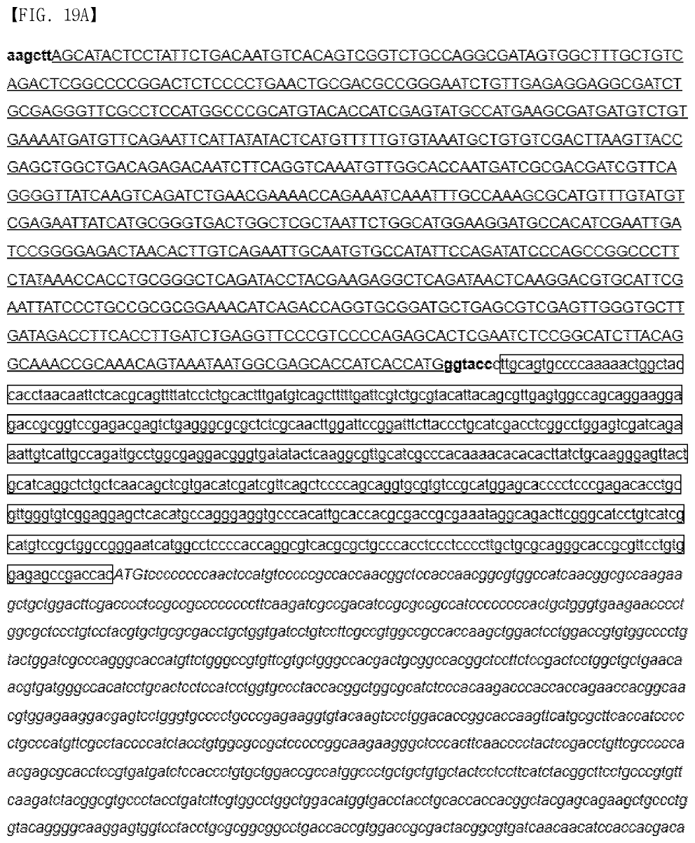

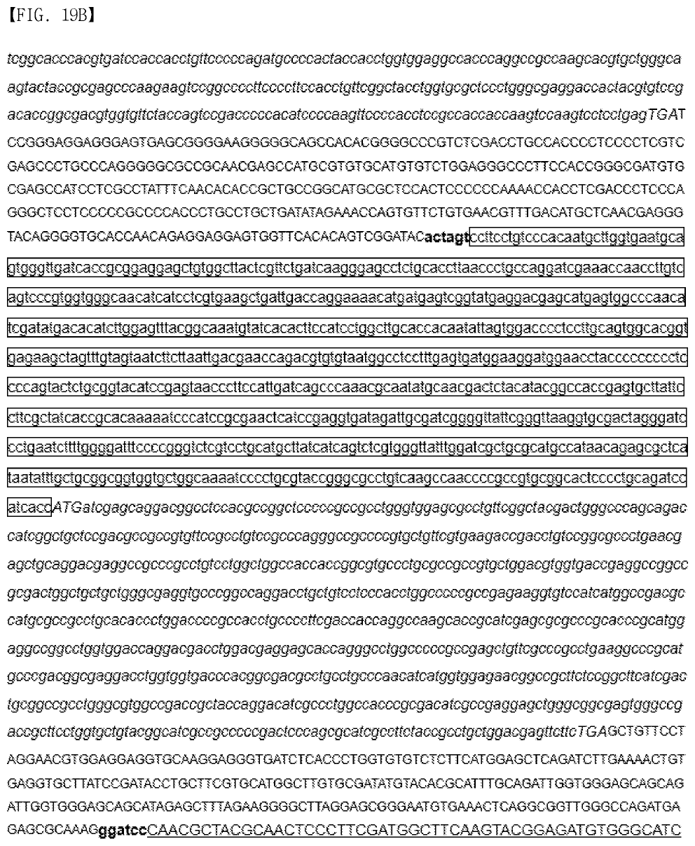

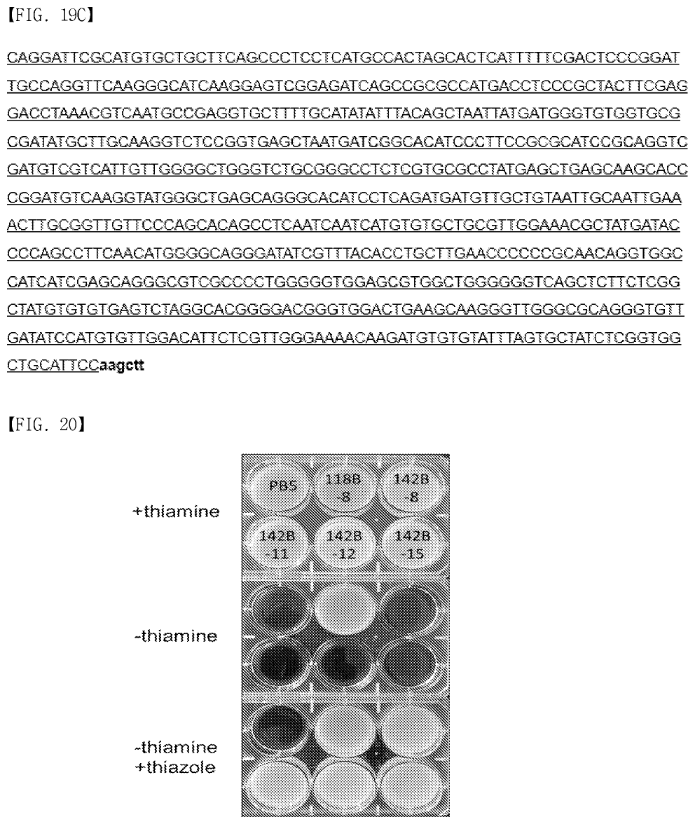

Upregulation of the endogenous FAD3 gene and overexpression of AtPDCT only resulted in small improvements in ALA accumulation, so we tested whether ALA biosynthesis could be further improved by the expression of heterologous FAD3. We chose to test FAD3A from Linum usitatissimum (flax), which has been shown previously to have desaturase activity in Prototheca moriformis. The sequence of the transforming construct pPB0142, targeting LuFAD3A and neoR expression to the THI4 locus is provided in FIG. 19. Restriction sites are indicated with bold, lowercase text. HindIII sites delimit the 5′ and 3′ ends of the transforming DNA. KpnI, SpeI, and BamHI sites separate the LuFADA expression module and the neoR expression cassette. Underlined sequences represent THI4 genomic DNA targeting integration at the THI4 locus via homologous recombination. Proceeding from 5′ to 3′, the selection cassette contains the ApSAD2 promoter (lowercase, boxed text), driving the expression of codon-optimized LuFAD3A. The initiator ATG and terminator TGA for LuFAD3A are indicated by uppercase italics, while the coding region is indicated with lowercase italics. The ApFBA1 terminator region is indicated by small capitals. Lowercase, boxed text delineates the ApPGK1 promoter, driving the expression of codon-optimized neoR, which confers resistance to the antibiotic G418. The initiator ATG and terminator TGA for neoR are indicated by uppercase italics, while the coding region is indicated with lowercase italics. The ApPGK1 terminator region is indicated by small capitals.

pPB0142 was transformed into strain 118B-8, and primary transformants were selected on G418-containing growth media supplemented with sucrose and without thiamine. Colonies were clonally purified and insertion of the constructs at the THI4 locus was verified by PCR amplification and sequencing of the regions flanking the integration site. We also verified that strains containing both constructs pPB0118 and pPB0142 disrupted both alleles of the THI4 locus, rendering them unable to grow without supplementation with hydroxymethyl thiazole (FIG. 20).

As shown in FIG. 20, Thiamine prototrophy is observed in strain 118B-8, expressing AtTHIC, targeted to one allele of THI4. Disruption of the second THI4 allele by pPB0142 renders the transformants thiazole auxotrophs.

Freeze-dried microalgal cell pellets were sent to Microbial ID (Delaware, USA) for fatty acid methyl ester analysis (FAME) and the profiles of lipids from shake flask assays of representative strains containing the pPB0142 construct are shown in Table 5. ALA accumulation increased from 5.4% in the 118B-8 parent to up to 8.7% in strain 142B-11, indicating that expression of LuFAD3A during lipid production enhanced desaturation of C18:2. The omega-6:omega-3 ratio was reduced to 4, similar to the original ratio observed in the parent strain 41-3 (see Table 4), but with a 5-fold increase in omega-3 fatty acids and a 3-fold improvement in overall PUFA compared to the wild-type strain.

TABLE 5
Fatty acid profiles as a percentage of total fatty
acids for strain 118B-8 transformed with pPB0142.

| strain | PB5 | 118B-8 | 142B-8 | 142B-11 |
|---|---|---|---|---|
| C14:0 myristic | 2.0 | 2.5 | 2.3 | 2.2 |
| C16:0 palmitic | 12.0 | 11.8 | 12.5 | 12.0 |
| C16:1n-7 palmitoleic | 0.4 | 0.3 | 0.4 | 0.4 |
| C18:0 stearic | 2.5 | 1.8 | 1.9 | 1.8 |
| C18:1n-9 oleic | 67.9 | 44.1 | 39.9 | 39.2 |
| C18:2n-6 linoleic LA | 13.5 | 34.0 | 34.4 | 34.6 |
| C18:3n-3 α-linolenic | 1.8 | 5.4 | 8.2 | 8.7 |
| omega-6:omega-3 | 7.6 | 6.3 | 4.2 | 4.0 |
| % PUFA | 15.3 | 39.4 | 42.6 | 43.3 |

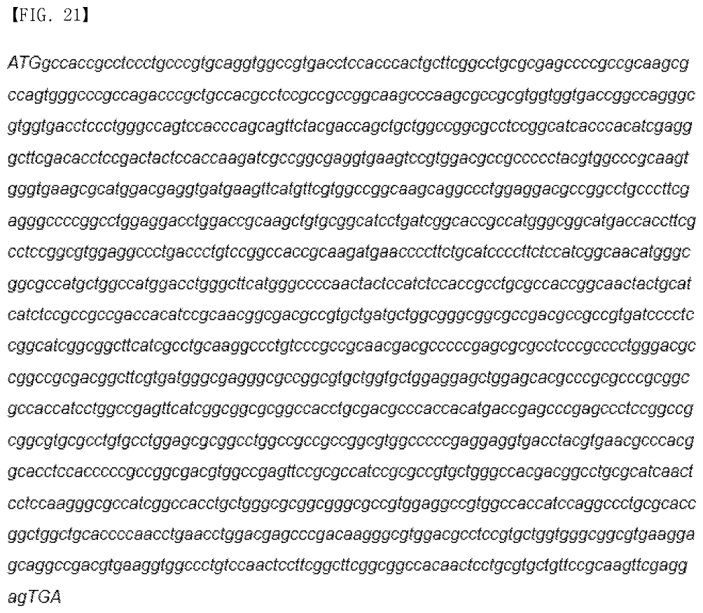

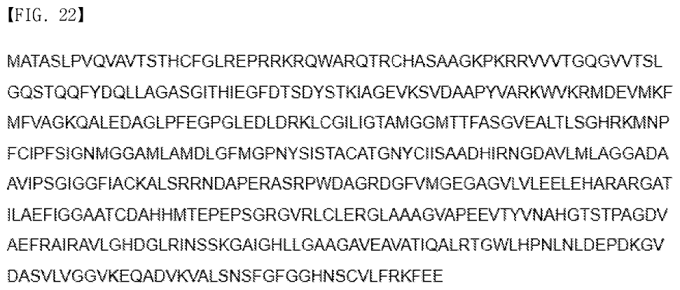

We aim to produce a nutritionally superior oil from A. protothecoides PB5, with very low saturates and a 1:1 ratio of omega-6: omega-3 fatty acids. Optimization of C18:2 and C18:3 α levels may be achieved by modulating the expression of AtPDCT and LuFAD3A. The main saturated fatty acids in wild-type A. protothecoides oil are C14:0, C16:0, and C18:0 (see Tables 3, 4 & 5). Levels of C14:0 and C16:0 can be reduced by over-expressing the native KASII gene, encoding beta-ketoacyl-ACP synthase II, which extends C16:0 to C18:0. The coding sequence of the Auxenochlorella KASII gene, optimized for translation, is shown in FIG. 21 and the corresponding amino acid sequence is detailed in FIG. 22.

### REFERENCES

1. Single cell oils. (AOCS Press, 2010).
2. Zaimes, G. G. & Khanna, V. Environmental sustainability of emerging algal biofuels: A comparative life cycle evaluation of algal biodiesel and renewable diesel. Environ. Prog. Sustain. Energy 32, 926-936 (2013).
3. Bhujade, R., Chidambaram, M., Kumar, A. & Sapre, A. Algae to Economically Viable Low-Carbon-Footprint Oil. Annu. Rev. Chem. Biomol. Eng. 8, 335-357 (2017).
4. Huang, Y.-S. & Sinclair, A. Lipids in Infant Nutrition. (AOCS Publishing, 1998). doi:10.1201/9781439831953.
5. Naguib, Y. M. A. Antioxidant Activities of Astaxanthin and Related Carotenoids. J. Agric. Food Chem. 48, 1150-1154 (2000).
6. Schmidt, I. et al. Biotechnological production of astaxanthin with Phaffia rhodozyma/Xanthophyllomyces dendrorhous. Appl. Microbiol. Biotechnol. 89, 555-571 (2011).
7. Elwan, H. a. M., Elnesr, S. S., Abdallah, Y., Hamdy, A. & El-Bogdady, A. H. Red yeast (Phaffia rhodozyma) as a source of Astaxanthin and its impacts on productive performance and physiological responses of poultry. Worlds Poult. Sci. J. 75, 273-284 (2019)
8. Nicolaides, N. (1974). Skin lipids: their biochemical uniqueness. Science 186, 19-26
9. Amarowicz, R. (2009). Squalene: a natural antioxidant? Eur. J. Lipid Sci. Technol. 111, 411-412
10. Günes, F. E. (2013). Medical use of squalene as a natural antioxidant. Clin. Exp. Health Sci. 3, 220-228
11. Kim, S. K., and Karadeniz, F. (2012). “Biological importance and applications of squalene and squalane,” in Advances in Food and Nutrition Research, Vol. 65. ed S. K. Kim (Walthum, MA: Academic Press), 223-233
12. Huang, Z. R., Lin, Y. K., and Fang, J. Y. (2009). Biological and pharmacological activities of squalene and related compounds: potential uses in cosmetic dermatology. Molecules 14, 540-554
13. Popa, O., Băbeanu, N. E., Popa, I., Nită, S., and Dinu-Pârvu, C. E. (2015). Methods for obtaining and determination of squalene from natural sources. Biomed. Res. Int. 2015:367/202
14. Aioi, A., Shimizu, T., and Kuriyama, K. (1995). Effect of squalene on superoxide anion generation induced by a skin irritant, lauroylsarcosine. Int. J. Pharm. 113, 159-164
15. Budiyanto, A., Ahmed, N. U., Wu, A., Bito, T., Nikaido, O., Osawa, T., et al. (2000). Protective effect of topically applied olive oil against photocarcinogenesis following UVB exposure of mice. Carcinogenesis 21, 2085-2090
16. Smith, T. J. (2000). Squalene: potential chemopreventive agent. Expert Opin. Invest. Drugs 9, 1841-1848
17. Kopicová, Z., and Vavreinová, S. (2007). Occurrence of squalene and cholesterol in various species of Czech freshwater fish. Czech. J. Food Sci. 25, 195-201
18. Del Giudice, G., Fragapane, E., Bugarini, R., Hora, M., Henriksson, T., Palla, E., et al. (2006). Vaccines with the MF59 adjuvant do not stimulate antibody responses against squalene. Clin. Vaccine Immunol. 13, 1010-1013
19. Pasquale, A. D., Preiss, S., Silva, F. T. D., and Garçon, N. (2015). Vaccine adjuvants: from 1920 to 2015 and beyond. Vaccines 3, 320-343
20. Ivanova, S., Tonchev, V., Yokoi, N., Yappert, M. C., Borchman, D., and Georgiev, G. A. (2015). Surface properties of squalene/meibum films and NMR confirmation of squalene in tears. Int. J. Mol. Sci. 16, 21813-21831
21. Rosales-Garcia, T., Jimenez-Martinez, C., and Dávila-Ortiz, G. (2017). Squalene extraction: Biological sources and extraction methods. Int. J. Environ. Agric. Biotechnol. 2, 1662-1670
22. Global Market Insights (2016). Squalene Market Size by Source, by Application, Industry Analysis Report, Regional Outlook, Application Potential, Price Trends, Competitive Market Share & Forecast, 2015-2022
23. Czaplicki, S., Ogrodowska, D., Zadernowski, R., and Derewiaka, D. (2012). Characteristics of biologically-active substances of amaranth oil obtained by various techniques. Pol. J. Food Nutr. Sci. 62, 235-239
24. Xiao Y, Lu Y, Dai J and Wu Q (2015) Industrial fermentation of Auxenochlorella protothecoides for production of biodiesel and its application in vehicle diesel engines. Front. Bioeng. Biotechnol. 3:164.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. Mutant microalgae to produce oil containing fatty acids, carotenoids, and/or terpenoids of an altered profile with disruption or ablation of one or more alleles of an endogenous polynucleotide or comprising an exogenous gene.

18. The microalgal mutant of claim 17, wherein the microalgal mutant is characterized in that at least one of the alleles LCYE-1 and LCYE-2 of lycopene cyclase epsilon are knocked out, or at least one of alleles SQE-1 and SQE-2 of squalene epoxidase are knocked out, or the native FAD3 promoter is replaced with a stearoyl-ACP desaturase (SAD2) promoter or the native FAD3 promoter is replaced with the promoter from the A. protothecoides FATA gene, encoding acyl-ACP thioesterase.

19. The microalgal mutant of claim 17, wherein the microalgal mutant is characterized in that one or more of the alleles of exogenous beta-ketolase 1 gene (CrBKT1) is knocked in.

20. The microalgal mutant of claim 17, wherein the microalga is Auxenochlorella protothecoides.

21. An oil produced by the microalgal mutant of claim 17.

22. Composition comprising a microalgal mutant of claim 17, a culture thereof, or oil of claim 21.

23. The composition of claim 22, wherein the composition is a cosmetic composition, a food composition, a composition for a food additive, a feed composition, a composition for a feed additive, a pharmaceutical composition, a raw material composition for food, a raw material composition for feed, a raw material composition for pharmaceutics or a raw material composition for cosmetics.

24. The microalgal mutant of claim 17, wherein a zeaxanthin has a percent (w/w) of zeaxanthin 2-3-fold higher compared to the wild-type microalgae and the zeaxanthin is present as a major carotenoid.

25. The microalgal mutant of claim 24, wherein the percent (w/w) of zeaxanthin produced ranges between 40 to 90% of total identified carotenoids.

26. The microalgal mutant of claim 17, wherein the oil contains a mixture of 4-keto lutein and astaxanthin, and wherein the astaxanthin is present as a major carotenoid.

27. The microalgal mutant of claim 26, wherein the percent (w/w) of keto carotenoids produced ranges between 20-90% of total identified carotenoids.

28. The microalgal mutant of claim 17, wherein the oil contains squalene.

29. The microalgal mutant of claim 17, wherein an omega-6 fatty acids and an omega-3 fatty acids have a weight ratio of omega-6 fatty acids to omega-3 fatty acids in the oil that is low compared to the oil produced from wild type microalgae.

30. The microalgal mutant of claim 29, wherein the weight ratio of omega-6 to omega-3 in the oil ranges from 1:1 to 5:1 compared to the oil produced from wild type microalgae which is 7:1.

31. The microalgal mutant of claim 17, wherein the omega-3 fatty acids increased 3-5-fold and the overall polyunsaturated fatty acids increased 2-3-fold compared to the wild-type strain.

32. The microalgal mutant of claim 17, wherein the microalgal mutant is characterized in that one or more of the alleles of the lycopene cyclase epsilon LCYE-1 gene, lycopene cyclase epsilon LYCE-2 gene are knocked out.

33. The microalgal mutant of claim 17, wherein the microalgal mutant is characterized in that one or more of the alleles of the squalene epoxidase SQE-1 gene, or squalene epoxidase SQE-2 gene are knocked out.

34. The microalgal mutant of claim 17, wherein the microalgal mutant is characterized in that the native FAD3 promoter is replaced with a stearoyl-ACP desaturase (SAD2) promoter or a promoter from the Auxenochorella protothecoides FATA gene encoding acyl-ACP thioesterase.

35. The microalgal mutant of claim 17, with modified profiles of omega-3 fatty acids, omega-6 fatty acids, lutein, zeaxanthin, astaxanthin, 4-keto-lutein, or squalene, wherein the microalgal mutant is a mutant of Auxenochlorella protothecoides, wherein the microalgal mutant comprises a knock-out of at least one allele of lycopene cyclase epsilon LCYE-1, lycopene cyclase epsilon LYCE-2, squalene epoxidase SQE-1, or squalene epoxidase SQE-2, or a replacement of a native FAD3 promoter by a FATA gene promoter from Auxenochorella protothecoides or a stearoyl-ACP desaturase (SAD2) promoter from Auxenochorella protothecoides.
